(19) United States
(12) Patent Application Publication
(10) Pub. No.: US 2014/0256695 A1
(43) Pub. Date: Sep. 11, 2014

(54) INJECTABLE FILLER

(71) Applicants: Phi Nguyen, Houston, TX (US); Loc Phan, San Jose, CA (US); Bao Tran, Saratoga, CA (US); Thuan Nguyen, Houston, CA (US)

(72) Inventors: Phi Nguyen, Houston, TX (US); Loc Phan, San Jose, CA (US); Bao Tran, Saratoga, CA (US); Thuan Nguyen, Houston, CA (US)

(21) Appl. No.: 13/872,071

(22) PCT Filed: Nov. 11, 2012
(86) PCT No.: PCT/US2012/064586

§ 371 Date: Apr. 27, 2013

Related U.S. Application Data
(63) Continuation-in-part of application No. 13/301,785, filed on Nov. 22, 2011, now abandoned.
(60) Provisional application No. 61/558,669, filed on Nov. 11, 2011.

(51) Int. Cl. A61L 27/26; A61L 27/54; A61L 27/52; A61L 27/50

(52) U.S. Cl. CPC A61L 27/26; A61L 27/50; A61L 27/54; A61L 27/52
USPC 514/180; 514/772.7; 514/626; 514/458

(57) ABSTRACT

Systems and method are disclosed for forming a biocompatible cross-linked polymer having an interpenetrating polymer network (IPN) by cross-linking a heteropolysaccharide to form a single cross-linked material; and performing one or more additional cross-linkings on the single cross-linked material to form a multiple cross-linked material, wherein the multiple cross-linked material has one or more IPN regions resisting biodegradation in a human body than the single cross-linked material and one or more single cross-linked extensions radiating out from the IPN, wherein the combination of the IPN and the extension provide biodegradation resistance, soft touch feeling, and ease of insertion into the human body.

This application is a US national filing of PCT Application PCT/US2012/064586 which claims priority to Provisional Application Ser. No. 61/558,669 filed Nov. 11, 2011 and Utility application Ser. No. 13/301,785, filed Nov. 22, 2011, the contents of which are incorporated by reference.

### BACKGROUND

The present invention relates to biocompatible viscoelastic polymeric gel slurries, methods for their preparation and formulations containing them.

As a person age, facial rhytids (wrinkles) and folds develop in respond to the loss of facial fat and the decrease of the skin elasticity. Physicians have over the years tried various methods and materials to combat the facial volume loss of the soft tissue of the face. One of the most common methods is autologous fat transfer. Using this surgical method, a person's own fat is harvested from a different part of the body such as the abdomen, and then the fat is processed and prepared for injection into the dermal and soft tissue areas of the face that is requiring the volume restoration to alleviate the wrinkles and folds to achieve a more youthful appearance. Autologous fat transfer has good desirable results, however, this surgical technique is costly, painful, time consuming, has a long recovery time for the patient, and is associated with complications associated with any surgical procedure.

### SUMMARY

There are a number of aspects that will be detailed below:

I. Serial Cross-Linking

II. HA Molecular Weight Manipulation

III. Free Radical Scavengers: Vitamins, Enzymes and similar

IV. Anti-hyaluronidase and Anti-Elastase

### Serial Cross-Linking

In one aspect, systems and methods are disclosed for cosmetic augmentation of soft tissue using cross-linked HA that had been optimized for

1. ease of product delivery,

2. local tissue compliant,

3. greater cohesiveness to control migration of the implant material and

4. bio-degradation profile.

The use of a particularly cross linked HA, and cross linked by forming regions of interpenetrating network (IPN) of cross linked HA by further crosslinking them. The IPN configuration gives this cross linked HA those utilities unique for this cosmetic augmentation application. The IPN core (imagine a tapioca ball) is more resistance to biodegradation in a human body than the single cross-linked material normalized for the same cross linking level. Furthermore, varying physical properties that continuously changes radiating out from the core makes the polymer tough and at the same time compliant with the local tissue for better tissue/device biocompatibility and feels more natural to the touch.

The above HA cross linking method optimized for cosmetic augmentation in certain cases may need to control delivered pharmaceutical substances to modulate local tissue response to the polymer. The pharmaceutical component makes up the multi-phase mixture with the other phase being the cross linked HA polymer

Implementations of the above aspects may include one or more of the following. The system is biocompatible and performs controlled drug releases at strategic timing to coincide with key physiological events. For example, a fast drug release profile and no delay would be well suited for the controlled release of an anesthetic such as lidocane to relieve acute pain experienced by the patient associated with the surgical procedure. The system is also capable of a medium release profile and a medium delay of a corticosteroid or steroid such as dexamethasone or triamcinolone to co-inside with a physiological inflammatory foreign body reaction. The system can also be customized to have a medium to slow release profile and a longer delay before starting the release of an antiproliferative drug such as paclitaxel, serolimas or 5-fluorouracil to stop uncontrolled healing and excessive remodeling causing unsightly scar formation or capsular formation.

1. Molecular Weight Manipulation

Another aspect of the present invention includes methods for optimizing biodegradation profiles and control migration of the implant material through the manipulation of various types molecular weight. The system optimizes biodegradation profiles and controls migration of the implant material. The system can be formulated around various types of molecular weights such as Mₙ, M_{w} and M_{z}, and their polydispersity index (PDI) to optimize the biodegradation profiles to be from hypervolumic to isovolumic to hypovolumic.

2. Free Radical Scavengers Vitamins and Enzymes

HA in the body is biodegraded by two major mechanisms: oxidative and hydrolytic. Inside the cell of mammals, the mechanism is enzymatic hydrolysis by three enzymes hyaluronidase (hyase), b-d-glucuronidase, and β-N-acetyl-hexosaminidase. and outside the cell the mechanism is oxidation by oxygen derived free radical, or sometimes, they are called reactive oxygen species (ROS). These are atoms or groups of atoms with an odd (unpaired) number of electrons and can be formed when oxygen interacts with certain molecules.

ROS are produced as a normal product of cellular metabolism. In particular, one major contributor to oxidative damage is hydrogen peroxide (H2O2), which is converted from superoxide that leaks from the mitochondria. Catalase and superoxide dismutase ameliorate the damaging effects of hydrogen peroxide and superoxide by converting these compounds into oxygen and water, benign molecules. However, this conversion is not 100% efficient, and residual peroxides persist in the cell. While ROS are produced as a product of normal cellular functioning, excessive amounts can cause deleterious effects. Memory capabilities decline with age, evident in human degenerative diseases such as Alzheimer's disease, which is accompanied by an accumulation of oxidative damage. Current studies demonstrate that the accumulation of ROS can decrease an organism's fitness because oxidative damage is a contributor to senescence. In particular, the accumulation of oxidative damage may lead to cognitive dysfunction, as demonstrated in a study in which old rats were given mitochondrial metabolites and then given cognitive tests. Results showed that the rats performed better after receiving the metabolites, suggesting that the metabolites reduced oxidative damage and improved mitochondrial function. Accumulating oxidative damage can then affect the efficiency of mitochondria and further increase the rate of ROS production. The accumulation of oxidative damage and its implications for aging depends on the particular tissue type where the damage is occurring. Additional experimental results suggest that oxidative damage is responsible for age-related decline in brain functioning. Older gerbils were found to have higher levels of oxidized protein in comparison to younger gerbils. Treatment of old and young mice with a spin trapping compound caused a decrease in the level of oxidized proteins in older gerbils but did not have an effect on younger gerbils. In addition, older gerbils performed cognitive tasks better during treatment but ceased functional capacity when treatment was discontinued, causing oxidized protein levels to increase.

Furthermore, once formed these highly reactive radicals can start a chain reaction. Their chief danger comes from the damage they can do when they react with important cellular components such as DNA, or the cell membrane. Cells may function poorly or die if this occurs. To prevent free radical damage the body has a defense system of antioxidants. The free radicals and the antioxidants react with one another readily and easily.

The degradation reaction by oxygen derived free radical of HA was the results of studies using the HA present in synovial fluids. It showed that the HA was readily degraded by super oxide free radicals. This reaction is most favorable in the case of secondary free radicals. Neutrophils (polymorphonuclear leukocytes) produced the type of oxygen derived free radicals that allowed it phagocytotically consumed HA molecules. These WBC's are by far the exclusive destroyers of HA by oxygen-derived free radical mechanism. Thus, an aspect of this invention is to quench the effect of the free radical before it degrades the HA using free radical scavengers such as antioxidant vitamins.

Antioxidants are intimately involved in the prevention of cellular damage—the common pathway for cancer, aging, and a variety of diseases. Antioxidants are molecules which can safely interact with free radicals and terminate the chain reaction before vital molecules are damaged. Although there are several enzyme systems within the body that scavenge free radicals, the principle micronutrient (vitamin) antioxidants are vitamin E, beta-carotene, and in the case of HA, vitamin C is the exception. Additionally, selenium, a trace metal that is required for proper function of one of the body's antioxidant enzyme systems, is sometimes included in this category. The body cannot manufacture these micronutrients so they must be supplied in the diet.

Following are example antioxidant vitamins, their roles and recommended daily dosages:
Vitamin E: d-alpha tocopherol. A fat soluble vitamin present in nuts, seeds, vegetable and fish oils, whole grains (esp. wheat germ), fortified cereals, and apricots. Current recommended daily allowance (RDA) is 15 IU per day for men and 12 IU per day for women.
Vitamin C: The exception in the case of HA as it is detrimental to the longevity of HA. However, vitamin C is ascorbic acid, and it is a water soluble vitamin present in citrus fruits and juices, green peppers, cabbage, spinach, broccoli, kale, cantaloupe, kiwi, and strawberries. The RDA is 60 mg per day. Intake above 2000 mg may be associated with adverse side effects in some individuals.
Vitamin A: Beta-carotene is a precursor to vitamin A (retinol) and is present in liver, egg yolk, milk, butter, spinach, carrots, squash, broccoli, yams, tomato, cantaloupe, peaches, and grains. Because beta-carotene is converted to vitamin A by the body there is no set requirement. Instead the RDA is expressed as retinol equivalents (RE), to clarify the relationship. (NOTE: Vitamin A has no antioxidant properties and can be quite toxic when taken in excess.)
Glutathione: (GSH) is a tripeptide with a gamma peptide linkage between the amine group of cysteine (which is attached by normal peptide linkage to a glycine) and the carboxyl group of the glutamate side-chain. It is an antioxidant, preventing damage to important cellular components caused by reactive oxygen species such as free radicals and peroxides. Thiol groups are reducing agents, existing at a concentration of approximately 5 mM in animal cells. Glutathione reduces disulfide bonds formed within cytoplasmic proteins to cysteines by serving as an electron donor. In the process, glutathione is converted to its oxidized form glutathione disulfide (GSSG), also called L(−)-Glutathione.
   Once oxidized, glutathione can be reduced back by glutathione reductase, using NADPH as an electron donor. The ratio of reduced glutathione to oxidized glutathione within cells is often used as a measure of cellular toxicity.
Uric Acid: It is the most important plasma antioxidant in humans, and a heterocyclic compound of carbon, nitrogen, oxygen, and hydrogen with the formula C5H4N4O3. It forms ions and salts known as urates and acid urates such as ammonium acid urate. Uric acid is a product of the metabolic breakdown of purine nucleotides. High blood concentrations of uric acid can lead to a type of arthritis known as gout. The chemical is associated with other medical conditions including diabetes and the formation of ammonium acid urate kidney stones.

Another aspect of this invention is the use of antioxidant enzymes to protect the longevity of HA. These enzymes can reduce the radicals and defend against ROS. They are: alpha-1-microglobulin, superoxide dismutases, catalases, lactoperoxidases, glutathione peroxidases and peroxiredoxins.

3. Anti-Hyaluronidase and Anti-Elastase

In respect to the field of cosmetic augmentation to bring back youthfulness to aging skin using cross-linked HA, an aspect of this invention uses hyaluronidase inhibitor (anti-HA) to prevent the depolymerization of HA, specifically by hyaluronidase, and to maintain the longevity of HA. Maintenance of HA longevity is important because it is directly related to the appearance of those unwanted wrinkles and the signs of aging.

HA is an important molecule to everything that lives on this earth. In that, it is a multifunctional high molecular weight polysaccharide found throughout the animal kingdom, especially in the extracellular matrix (ECM) of soft connective tissues. HA is thought to participate in many biological processes, and its level is markedly elevated during embryogenesis, cell migration, wound healing, malignant transformation, and tissue turnover. The enzymes that degrade HA, hyaluronidases (HAases) are expressed both in prokaryotes and eukaryotes. These enzymes are known to be involved in physiological and pathological processes ranging from fertilization to aging. Hyaluronidase-mediated degradation of HA increases the permeability of connective tissues and decreases the viscosity of body fluids and is also involved in bacterial pathogenesis, the spread of toxins and venoms, acrosomal reaction/ovum fertilization, and cancer progression. Furthermore, these enzymes may promote direct contact between pathogens and the host cell surfaces. Depolymerization of HA also adversely affects the role of ECM and impairs its activity as a reservoir of growth factors, cytokines and various enzymes involved in signal transduction. Inhibition of HA degradation therefore may be crucial in reducing disease progression and spread of venom/toxins and bacterial pathogens. Hyaluronidase inhibitors are potent, ubiquitous regulating agents that are involved in maintaining the balance between the anabolism and catabolism of HA. Hyaluronidase inhibitors could also serve as contraceptives and anti-tumor agents and possibly have antibacterial and anti-venom/toxin activities. Additionally, these molecules can be used as pharmacological tools to study the physiological and pathophysiological role of HA and hyaluronidases.

The mechanism of hyaluronidase in the degradation of HA generally follows five steps:

| | |
|---|---|
| Step 1. | The enzyme binds negatively charged |
| | substrate in the binding cleft |
| | using electropositive residues |
| | constituting the hydrophobic patch; |
| Step 2. | This is a catalytic step involving |
| | catalytic residues Asn 349, His |
| | 399, and Tyr 408 results in the |
| | cleaving of β 1-4 bond with the |
| | generation of disaccharide end product; |
| Step 3. | A hydrogen exchange between |
| | the enzyme and the water |
| | microenvironment in order to return |
| | the enzyme to its natural state |
| | ready for the next round of catalysis; |
| Step 4. | The irreversible step of the release |
| | of the disaccharide product by |
| | utilizing negative patch in the cleft, and finally |
| Step 5. | A translocation of the remaining |
| | HA by one disaccharide unit |
| | towards the substrate's reducing end. |

The Table below shows examples of hyaluronidase inhibitors

List of different class of hyaluronidase inhibitors

| Type of compound | Compounds |
|---|---|
| Alkaloids | Aristolochic acid, ajmaline, reserpine |
| Antioxidants | Ascorbic acid, NDGA, N-propyl gallate, BHT, chlorogenic acid, curcumin, tannic acid |
| Anti-inflammatory | Dexamethasone, indomethacin, sodium cromoglycate, salicylates, tranilast, sodium aurothiomalate, |
| drugs | myocrisin, gossypol, |
| Terpenoids/ | Flavone, Fenoprofen, Quercetin, Apigenin, Kaempferol, Silybin, Luteolin, Hesperidin, Triterpenes, |
| flavonoids | Rutin, Myricetin, Glycynhizin, Glycynbetinic acid |
| Synthetic | PS₅₃ (Hydroquinone-sulfonic acid-formaldehyde polymer), phosphorylated hesperidin, polymer of |
| compounds | poly (styrene-4-sulfonate), sodium cellulose sulfate, 1-tetradecane sulfonic acid, L-arginin derivatives, |
| | traxanox, norlignane, urolithin B, aescin, diphenylacrylic acids, diphenyl propionic acids, indole |
| | derivatives, chalcone derivatives. |
| Glycosaminoglycans | Heparin, heparan sulfate, dermatan sulfate, chondroitin sulfate (A, C, D), O-sulfated HA, linamarin, |
| and glycosides, | amygdalin, |
| Fatty acids | Saturated (C_{10:0} to C_{22:0}), cis-unsaturated fatty acids (C_{14:1} to C_{24:1}) |
| Polysaccharides/ | Chitosans, dextran sulfate, sodium alginate, planteose derivatives, hydrochinone digalactoside, |
| oligosaccharides | 2-hydroxyphenyl manolactobioside, sulphated neomycin, verbascose, lanostanoids |
| Other proteins | Withania somnifera glycoprotein (WSG), Serum hyaluronidase inhibitor |
| Other reagents | HCN, L-NAME, L-arginine, Guanidium HCl |

### BRIEF DESCRIPTION OF THE DRAWINGS

FIG. 1 shows an exemplary system to produce multiply cross-linked HA.

FIG. 2 shows another exemplary system to produce multiply cross-linked HA.

FIG. 3 shows an exemplary diagram of the resulting multiply cross-linked HA.

FIG. 4 shows an exemplary dual pump HA mixing method.

FIG. 5 shows an exemplary continuous pump HA mixing method.

FIG. 6 shows an exemplary peristaltic pump HA mixing method.

FIG. 7 shows a schematic representation of another embodiment of IPN HA.

### DESCRIPTION

First, the preparation of the hyaluronic acid is discussed, followed by the addition of additional chemicals to enhance the use of the hyaluronic for dermal or subdermal use is discussed.

Since the hyaluronan of a recombinant Bacillus cell is expressed directly to the culture medium, a simple process may be used to isolate the hyaluronan from the culture medium. First, the Bacillus cells and cellular debris are physically removed from the culture medium. The culture medium may be diluted first, if desired, to reduce the viscosity of the medium. Many methods are known to those skilled in the art for removing cells from culture medium, such as centrifugation or microfiltration. If desired, the remaining supernatant may then be filtered, such as by ultrafiltration, to concentrate and remove small molecule contaminants from the hyaluronan. Following removal of the cells and cellular debris, a simple precipitation of the hyaluronan from the medium is performed by known mechanisms. Salt, alcohol, or combinations of salt and alcohol may be used to precipitate the hyaluronan from the filtrate. Once reduced to a precipitate, the hyaluronan can be easily isolated from the solution by physical means. The hyaluronan may be dried or concentrated from the filtrate solution by using evaporative techniques known to the art, such as lyophilization or spray drying.

Molecular Weight

The content of hyaluronic acid may be determined according to the modified carbazole method (Bitter and Muir, 1962, Anal Biochem. 4: 330-334). Moreover, the number average molecular weight of the hyaluronic acid may be determined using standard methods in the art, such as those described by Ueno et al., 1988, Chem. Pharm. Bull. 36, 4971-4975; Wyatt, 1993, Anal. Chim. Acta 272: 1-40; and Wyatt Technologies, 1999, “Light Scattering University DAWN Course Manual” and “DAWN EOS Manual” Wyatt Technology Corporation, Santa Barbara, Calif.

In one embodiment, the hyaluronic acid, or salt thereof, of the one embodiment has a molecular weight of about 10,000 to about 10,000,000 Da. In a more preferred embodiment it has a molecular weight of about 25,000 to about 5,000,000 Da. In a most preferred embodiment, the hyaluronic acid has a molecular weight of about 50,000 to about 3,000,000 Da.

In another embodiment, the hyaluronic acid or salt thereof has a molecular weight in the range of between 300,000 and 3,000,000; preferably in the range of between 400,000 and 2,500,000; more preferably in the range of between 500,000 and 2,000,000; and most preferably in the range of between 600,000 and 1,800,000.

In yet another embodiment, the hyaluronic acid or salt thereof has a low number average molecular weight in the range of between 10,000 and 800,000 Da; preferably in the range of between 20,000 and 600,000 Da; more preferably in the range of between 30,000 and 500,000 Da; even more preferably in the range of between 40,000 and 400,000 Da; and most preferably in the range of between 50,000 and 300,000 Da.

### EXAMPLES

### Example 1

### Preparation of DVS Crosslinked Microparticles in Emulsion

This example illustrates the preparation of DVS-crosslinked microparticles. Sodium hyaluronate (HA, 580 kDa, 1.90 g) was dissolved in aqueous NaOH (0.2 M, 37.5 ml) by vigorous stirring at room temperature for 3 hours until a homogenous solution was obtained. Sodium chloride (0.29 g) was added and mixed shortly. Mineral oil (10.0 g) and ABIL® EM 90 surfactant (Cetyl PEG/PPG-10/1 Dimethicone, 1.0 g) were mixed by stirring.

Divinylsulfone (DVS, 320 microliter) was added to the aqueous alkaline HA-solution and mixed for 1 min. to obtain a homogeneous distribution in the aq. phase. The water phase was then added within 2 minutes to the oil phase with mechanical stirring at low speed. An emulsion was formed immediately and stirring was continued for 30 minutes at room temperature. The emulsion was left over night at room temperature. The emulsion was neutralized to pH 7.0 by addition of aq. HCI (4 M, approx. 2.0 ml) and stirred for approx. 40 min.

### Example 2

### Preparation of DVS Crosslinked Microparticles in Emulsion Neutralized with Use of pH Indicator

This example illustrates the preparation of DVS-crosslinked microparticles with neutralization using a pH indicator. Sodium hyaluronate (HA, 580 kDa, 1.88 g) was dissolved in aqueous NaOH (0.2 M, 37.5 ml) by vigorous stirring at room temperature for 2 hours until a homogenous solution was obtained. Bromothymol blue pH indicator (equivalent range pH 6.6-6.8) was added (15 drops, blue color in solution). Sodium chloride (0.25 g) was added and mixed shortly.

Mineral oil (10.0 g) and ABIL® EM 90 surfactant (Cetyl PEG/PPG-10/1 Dimethicone, 1.0 g) were mixed by stirring.

Divinylsulfone (DVS, 320 microliter) was added to the aqueous alkaline HA-solution and mixed very vigorously for 30 to 60 seconds to obtain a homogeneous distribution in the aq. phase. The water phase was then added within 30 sec. to the oil phase with mechanical stirring at 400 RPM. An emulsion was formed immediately and stirring was continued for 30 min. at room temperature. Neutralization was performed by addition of aq. HCI (4 M, 1.6 ml) and the emulsion was left at room temperature with magnetic stirring for 4 hours. The pH indicator present in the gel particles changed color to green. pH in the emulsion was measured by pH stick to 3-4. The emulsion was left in fridge overnight. The pH indicator present in the gel particles had changed to yellow.

### Example 3

### Phase Separation of Emulsion, Swelling and Isolation of Microparticles

This example illustrates the breakage of the W/O emulsion followed by phase separation and dialysis. The crosslinked HA microparticles were separated from the W/O emulsion by organic solvent extraction. The W/O emulsion (5 g) and a mixture of n-butanol/chloroform (1/1 v %, 4.5 ml) was mixed vigorously by whirl mixing in a test tube at room temperature. Extra mQ-water (20 ml) was added to obtain phase separation. The test tube was centrifuged and three phases were obtained with the bottom phase being the organic phase, middle phase of gel particles and upper phase of clear aqueous solution. The top and bottom phases were discarded and the middle phase of gel particles was transferred into a dialysis tube (MWCO 12-14,000, Diameter 29 mm, Vol/Length 6.4 ml/cm). The sample was dialyzed overnight at room temperature in MilliQ®-water. The dialysate was changed two more times and left overnight. The resulting gel was thick and viscous and had swelled to a volume of approximately 50 ml, which correlated to 0.004 g HA/cm³.

### Example 4

### Preparation of DVS Crosslinked Microparticles in Emulsion and Separation of Microparticles

This example illustrates the preparation of DVS-crosslinked HA microparticles. Sodium hyaluronate (HA, 580 kDa, 1.89 g) was dissolved in aqueous NaOH (0.2 M, 37.5 ml). Sodium chloride (0.25 g) was added and the solution was stirred by magnetic stirring for 1 hour at room temperature until a homogeneous solution was obtained. TEGOSOFT® M (10.0 g) oil and ABIL® EM 90 surfactant (Cetyl PEG/PPG-10/1 Dimethicone, 1.0 g) were mixed by stirring.

Divinylsulfone (DVS, 320 microliter) was added to the aqueous alkaline HA-solution and mixed for 1 min. to obtain a homogenoues distribution in the aq. phase. The water phase was then added within 2 min. to the oil phase with mechanical stirring (300 RPM). An emulsion was formed immediately and stirring was continued for 30 min. at room temperature.

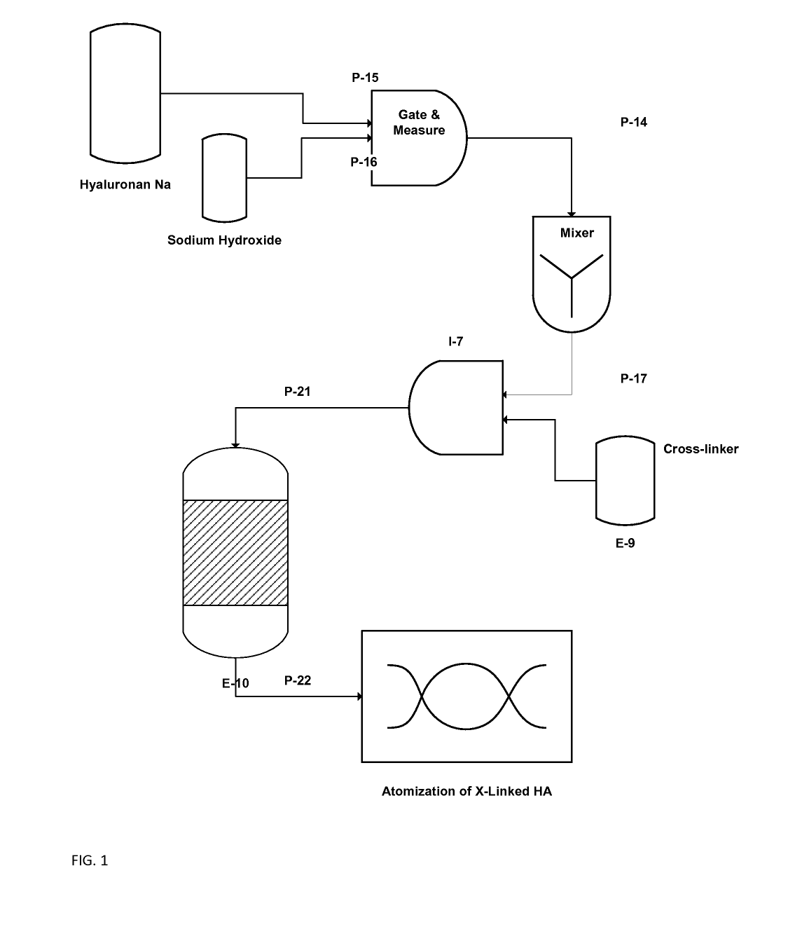

The emulsion was neutralized by addition of stociometric amounts of HCI (4 M, 1.8 ml) and stirred for approx. 40 min. The emulsion was broken by addition of a n-butanol/chloroform mixture (1:1 v %, 90 ml) and extra MilliQ®-water (100 ml) followed by magnetic stirring. The upper phase was separated in a volume of approx. 175 ml. The organic phase was mixed with mQ-water (30 ml) for a final washing. The combined water/gel phase (205 ml) were transferred to a dialysis tube (MWCO 12-14,000, Diameter 29 mm, Vol/Length 6.4 ml/cm) and dialysed against MilliQ®-water overnight at room temperature. The conductivity were decreased to 0.67 micro-Sievert/cm after subsequent change of water (3 times) and dialysis overnight (2 nights). The microparticles were assessed by microscopy (DIC 200×), see FIG. 1; the cross-section of one microparticle is indicated and labelled “21,587.92 nm”.

### Example 5

### Phase Separation of Emulsion and Isolation of Microparticles

This example illustrates the breakage of the W/O emulsion and isolation of the gel microparticles. The gel microparticles were separated from the W/O-emulsion by organic extractions. Examples of organic solvents which were used for this extraction were mixtures of butanol/chloroform in volume ratios (v %) of 75:20 to 20.80, respectively. The weight ratio (w %) of W/O emulsion to organic solvent was approximately 1:1.

Separation in small scale: The W/O emulsion (5 g) was weighed in centrifuge tubes (50 ml). A mixture of butanol/chloroform was prepared (1:1 v %) and from this mixture 4.5 ml was added (corresponds to 5 g) to the test tube. The test tube was carefully mixed to secure that all emulsion was dissolved. The test tube was mixed by Whirl mixing and left at room temperature for phase separation. Phase separation with water phase on top and organic phase at bottom with a white emulsion phase in between was often observed. Addition of more water and organic phases improved separation. The water phase was separated by decanting and further purified or characterized.

### Example 6

### Preparation of Water-in-Oil Emulsions

This example illustrates a composition in which the HA microparticles were formed. A hot/cold procedure can be used with incorporation of a cold water phase B into a hot oil phase, which will shorten the time of manufacture. A non-limiting example of formulation could be as follows:

### Phase A:

2.0% ABIL® EM 90 (cetyl PEG/PPG-10/1 dimethicone)
20.0% Mineral oil (or TEGOSOFT® M)

### Phase B:

0.5% Sodiumchloride
3.8% Hyaluronic acid
0.2 M NaOH (aq) up to 100%

### Phase C:

Approx. 0.6% Divinylsulfone

### Preparation:

1. Mix phase A at room temperature.
2. Phase B: Solubilize hyaluronic acid (Hyacare®) in aq. NaOH by stirring; then add NaCl and stir.
3. Add DVS to phase B and stir for 1 min.
4. Add phase B slowly to phase A with stirring.
5. Homogenise or stir for a short time and leave to react.
6. Stirring and swelling.
7. Continue stirring below 30° C.
8. Neutralize.

### Example 7

### Preparation and Separation of DVS Cross-Linked Microparticles

Sodium hyaluronate (HA, 580 kDa, 1.88 g) was dissolved in aqueous NaOH (0.2 M, 37.5 mL). Sodium chloride (0.25 g) was added and the solution was stirred by magnetic stirring for 1 hour at room temperature until a homogeneous solution was obtained. The oil: TEGOSOFT® M (10.0 g) and surfactant: ABIL® EM 90 (Cetyl PEG/PPG-10/1 Dimethicone, 1.0 g) was mixed by stirring. Divinylsulfone (DVS, 320 microliter) was added to the aqueous alkaline HA-solution and mixed for 1 min to obtain a homogenoues distribution in the aq. phase. The water phase was then added within 2 min to the oil phase with mechanical stirring (300 RPM). An emulsion was formed immediately and stirring was continued for 30 min at room temperature.

The emulsion was neutralized by addition of stociometric amounts of HCI (4 M, 1.8 mL) and stirred for approx. 40 min. The emulsion was transferred to a separation funnel, and broken by addition of a n-butanol/chloroform mixture (1:1 v %, 90 mL) and extra millliQ™-water (100 mL) followed by vigorous shaking. The upper phase was separated in a volume of approx. 175 mL. The organic phase was washed with millliQ™-water (100 mL). The combined water/gel phase was transferred to a dialysis tube (MWCO 12-14,000, Diameter 29 mm, Vol/Length 6.4 mL/cm) and dialysed against millliQ™-water overnight at room temperature. The conductivity was decreased to 10 micro-Sievert/cm after subsequent change of water (3 times) and dialysis overnight (2 nights).

### Example 8

### Washing Procedure to Purify Microparticles

This example illustrates the final isolation and purification of the microparticles.

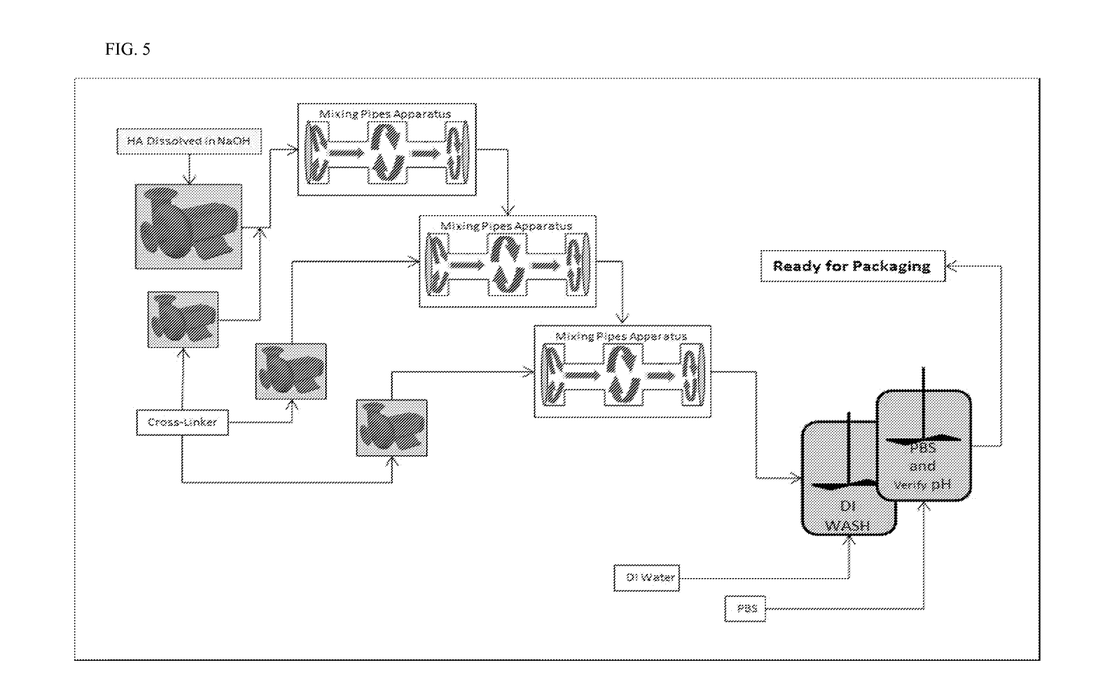

100 mL particles previously isolated were re-suspended in a Na 2 HPO 4/NaH 2 PO 4 buffer (0.15 M, 400 mL), and stirred slowly for ½ hour. The suspension stood at 5° C. for 2 hours and solidified oil droplets were removed. The solution was then filtered through a mesh and washed further with 2×50 mL buffer. Particles were allowed to drip-dry, before characterization (FIG. 5).

### Example 9

### Investigation of Rheological Properties of Microparticles

This example illustrates performance of rheological studies on particles. A particle sample is analyzed on an Anton Paar rheometer (Anton Paar GmbH, Graz, Austria, Physica MCR 301, Software: Rheoplus), by use of a 50 mm 2° cone/plate geometry. First the linear range of the visco-elastic properties G′ (Storage modulus) and G″ (Loss modulus) of the material is determined by an amplitude sweep with variable strain, γ. Secondary a Frequency sweep is made, and based on values of the visco-elastic values, G′ and G″, tan δ can be calculated as a value for week/strong gel behaviors.

### Example 10

### Investigation of Syringe ability Experiments on Texture Analyzer

This example illustrates performance of an investigation of force applied to inject at a certain speed, as a function of the homogeneity of the sample. A particle sample is transferred to a syringe applied with a needle, either 27G×½″, 30G×½″, and is set in a sample rig, in a texture analyzer (Stable Micro Systems, Surrey, UK, TA.XT Plus, SoftWare: Texture Component 32). The test is performed with an injection speed at 12.5 mm/min., over a given distance.

### Example 11

### Preparation of DVS-Crosslinked HA Hydrogels

This example illustrates the preparation of DVS-cross-linked HA hydrogels with concomitant swelling and pH adjustment.

Sodium hyaluronate (HA, 770 kDa, 1 g) was dissolved into 0.2M NaOH to give a 4% (w/v) solution, which was stirred at room temperature, i.e. about 20° C., for 1 h. Three replicates were prepared. Divinylsulfone (DVS) was then added to the HA solutions in sufficient amount to give HA/DVS weight ratios of 10:1, 7:1, and 5:1, respectively. The mixtures were stirred at room temperature for 5 min and then allowed to stand at room temperature for 1 h. The gels were then swollen in 160 mL phosphate buffer (pH 4.5 or 6.5) for 24 h, as indicated in Table 1.

TABLE 1
Conditions for DVS-HA hydrogel preparation.

| | HA/DVS | |
|---|---|---|
| Gel ID | weight ratio | Phosphate buffer used for swelling |
| 1 | 5:1 | 160 ml (pH 4.5) |
| 2 | 7:1 | 80 ml (pH 4.5) + 80 ml (pH 6.5) |
| 3 | 10:1 | 160 ml (pH 6.5) |

The pH of the gels was stabilized during the swelling step. After swelling, any excess buffer was removed by filtration and the hydrogels were briefly homogenized with an IKA® ULTRA-TURRAX® T25 homogenizer (Ika Labortechnik, DE). The volume and pH of the gels were measured (see Table 2).

TABLE 2
Characteristics of DVS-HA hydrogels.

| | HA/DVS | Vol of | HA | | | |
|---|---|---|---|---|---|---|
| Gel | weight | swollen | Conc. | | | |
| ID | ratio | gel | (w/v) | pH | Appearance | Softness |
| 1 | 5:1 | 70 mL | 1.4% | 7.1 | Transparent, | + |
| | | | | | homogenous | |
| 2 | 7:1 | 70 mL | 1.4% | 7.6 | Transparent, | ++ |
| | | | | | homogenous | |
| 3 | 10:1 | 70 mL | 1.4% | 7.5 | Transparent, | +++ |
| | | | | | homogenous | |

The pH of the hydrogels ranged from 7.1 to 7.6 (table 2), which confirms that the swelling step can be utilized to adjust the pH in this process. All the hydrogels occupied a volume of 70 mL, which corresponds to a HA concentration of ca. 1.4% (w/v). They were transparent, coherent and homogenous. Softness increased with decreasing cross-linking degree (Table 2).

### Example 12

### Preparation of Homogenous DVS-Crosslinked HA Hydrogels

This example illustrates the preparation of highly homogenous DVS-cross-linked HA hydrogels.

Sodium hyaluronate (770 kDa, 2 g) was dissolved into 0.2M NaOH with stirring for approx. 1 hour at room temperature to give a 8% (w/v) solution. DVS was then added so that the HA/DVS weight ratio was 7:1. After stirring at room temperature for 5 min, one of the samples was heat treated at 50° C. for 2 h without stirring, and then allowed to stand at room temperature overnight. The resulting cross-linked gel was swollen into 200 ml phosphate buffer (pH 5.5) 37° C. for 42 or 55 h, and finally washed twice with 100 ml water, which was discarded. Volume and pH were measured, as well as the pressure force necessary to push the gels through a 27G*½ injection needle (see Table 3).

The cross-linked HA hydrogel prepared according to this example exhibited a higher swelling ratio and an increased softness compared to a control hydrogel which was not heat treated (Table 3). The pressure force applied during injection through a 27G*½ needle was more stable than that of the latter sample, indicating that the cross-linked HA hydrogel is more homogenous.

TABLE 3
Characteristics of DVS-cross-linked HA hydrogels.

| | | | | | | | Stability of |
|---|---|---|---|---|---|---|---|
| | | | HA | | | | pressure |
| | Heat | Volume of | concentration | | | | force during |
| Gel ID | treated | swollen gel | (w/v) | pH | Appearance | Softness | injection |
| 1 | Yes | 145 mL | 1.4% | 6.1 | Transparent, | +++ | +++ |
| | | | | | homogenous | | |
| 2 | No | 90 mL | 1.1% | 6.7 | Transparent, | + | + |
| | | | | | homogenous | | |

### Example 13

### Biostability of DVS-Crosslinked HA Hydrogels

This example illustrates the in vitro biostability of DVS-cross-linked HA hydrogels using enzymatic degradation.

A bovine testes hyaluronidase (HAase) solution (100 U/mL) was prepared in 30 mM citric acid, 150 mM Na₂ HPO₄, and 150 mM NaCl (pH 6.3). DVS-HA cross-linked hydrogel samples (ca. 1 mL) were placed into safe-lock glass vials, freeze-dried, and weighed (W₀; Formula 1). The enzyme solution (4 mL, 400 U) was then added to each sample and the vials were incubated at 37° C. under gentle shaking (100-200 rpm). At predetermined time intervals, the supernatant was removed and the samples were washed thoroughly with distilled water to remove residual salts, they were then freeze-dried, and finally weighed (Wₜ; Formula 1).

The biodegradation is expressed as the ratio of weight loss to the initial weight of the sample (Formula 1). Weight loss was calculated from the decrease of weight of each sample before and after the enzymatic degradation test. Each biodegradation experiment was repeated three times. DVS-HA hydrogels prepared as described in example 2 (‘Heated’) were compared to DVS-HA hydrogels which had not been heat treated (‘Not heated’). For both types of gel, degradation was fast during the first four hours, and then proceeded slower until completion at 24 h. Importantly there was a significant variation of the weight loss values for the samples which had not been heated as compared to the hydrogel prepared with a heating step as described in example 2. This clearly illustrates that a highly homogenous DVS-cross-linked HA hydrogel is obtained by using the process described in example 2.

### Example 14

### Preparation of Water-in-Oil Emulsions for Cosmetics

In this and in the following example, DVS-crosslinked HA hydrogels were formulated into creams and serums, that when applied to the skin increase the skin moisturization and elasticity, and provide immediate anti-aging effect, as well as film-forming effect

A typical formulation of a water-in-oil (w/o) emulsion containing 2% DVS-cross-linked HA. Each phase (A to E) was prepared separately by mixing the defined ingredients (see Table 4). Phase B was then added to phase A under stirring with a mechanical propel stirring device and at a temperature less than 40° C. Phase C was then added followed by phase D and finally phase E under stirring. Formulations were also made, wherein the HA hydrogel concentration was 4%, 6% and 8%, respectively, in Phase D, to give a range of w/o formulations.

TABLE 4

| | | Proportion | |
|---|---|---|---|
| Phase | Ingredient | (w/w) | Function |
| A | Cyclopentasiloxane, dimethicone | 10% | Emollient |
| | Cyclopentasiloxane | 15% | Emollient |
| | Cyclopentasiloxane and | 4% | Emulsifier |
| | PEG/PPG- | | |
| | 20/15 Dimethicone | | |
| | Hydrogenated polydecene | 8% | Emollient |
| B | Water | 49.3% | |
| | Sodium chloride | 0.2% | |
| C | Tocopheryl acetate | 0.5% | Antioxidant |
| D | DVS Cross-linked sodium | 2% | |
| | hyaluronate | | |
| | Water | 10% | |
| E | Phenoxyethanol, | 1% | Preservative |
| | ethylhexylglycerin | | |

Another typical formulation of a w/o-emulsion containing 2% DVS-crosslinked HA is shown in table 5. Each phase (A to F) in table 5 was prepared separately by mixing the defined ingredients (see Table 5). Phase B was mixed with phase A and the resulting oil phase was heated at 75° C. Phase C was also heated to 75° C. The oil phase was added to phase C at 75° C. under stirring with a mechanical propel stirring device. The emulsion was then cooled down to less than 40° C., after which phase D was added, followed by phase E and finally phase F under stirring. Formulations were also made, wherein the HA hydrogel concentration was 4%, 6% and 8%, respectively, in Phase E, to give a range of w/o formulations.

TABLE 5

| | | Proportion | |
|---|---|---|---|
| Phase | Ingredient | (w/w) | Function |
| A | Hydrogenated polydecene | 18% | Emollient |
| | Acrylates/C10-30 alkyl acrylate | 1% | Thickener |
| | crosspolymer | | |
| B | Sodium cocoyl Glutamate | 10% | Emulsifier |
| C | Aqua | 53.5% | |
| | Distarch Phosphate | 2% | Texture agent |
| D | Tocopheryl acetate | 0.5% | Antioxidant |
| | Cyclopentasiloxane, dimethicone | 2% | Feeling and |
| | | | spreading |
| | | | agent |
| E | Cross-linked sodium | 2% | |
| | hyaluronate | | |
| | Aqua | 10% | |
| F | Phenoxyethanol, | 1% | Preservative |
| | ethylhexylglycerin | | |

### Example 15

### Preparation of Silicone Serums

A typical formulation of a silicone serum containing 2% DVS-cross-linked HA was prepared as shown in table 6. All ingredients were mixed at the same time under very high stirring and at less than 40° C. (see table 6). Formulations were also prepared, wherein the HA hydrogel concentration was 4%, 6% and 8%, respectively, to give a range of serums.

TABLE 6

| | Proportion | |
|---|---|---|
| Ingredient | (w/w) | Function |
| Cyclopentasiloxane | 60% | Line blurring |
| C30-45 Alkyl Cetearyl | | effect, |
| Dimethicone | | thickener, vehicle |
| Crosspolymer | | |
| Cyclopentasiloxane | 34.5% | Vehicle, emollient |
| Polymethylsilsesquioxane | 2.5% | Soft powdery feel |
| Cross-linked sodium | 2% | |
| hyaluronate | | |
| Phenoxyethanol, | 1% | Preservative |
| ethylhexylglycerin | | |

### Example 16

### pH Equilibration During Swelling; a Kinetics Study

A kinetics study showed that DVS cross-linked HA hydrogels with neutral pH are obtained after swelling in phosphate buffer (pH 7.0) for 8 to 14 hours, depending on the degree of cross-linking A set of DVS cross-linked HA hydrogels was prepared as described in the above, using from 4 to 8% HA solution, and using various amounts of DVS cross-linker, as indicated in Table 7.

TABLE 7

| | Initial | HA HA/DVS |
|---|---|---|
| | concentration | weight |
| Entry | (w/v) | ratio |
| 1 | 4% | 2.5:1 |
| 2 | 6% | 15:1 |
| 3 | 8% | 15:1 |
| 4 | 6% | 10:1 |

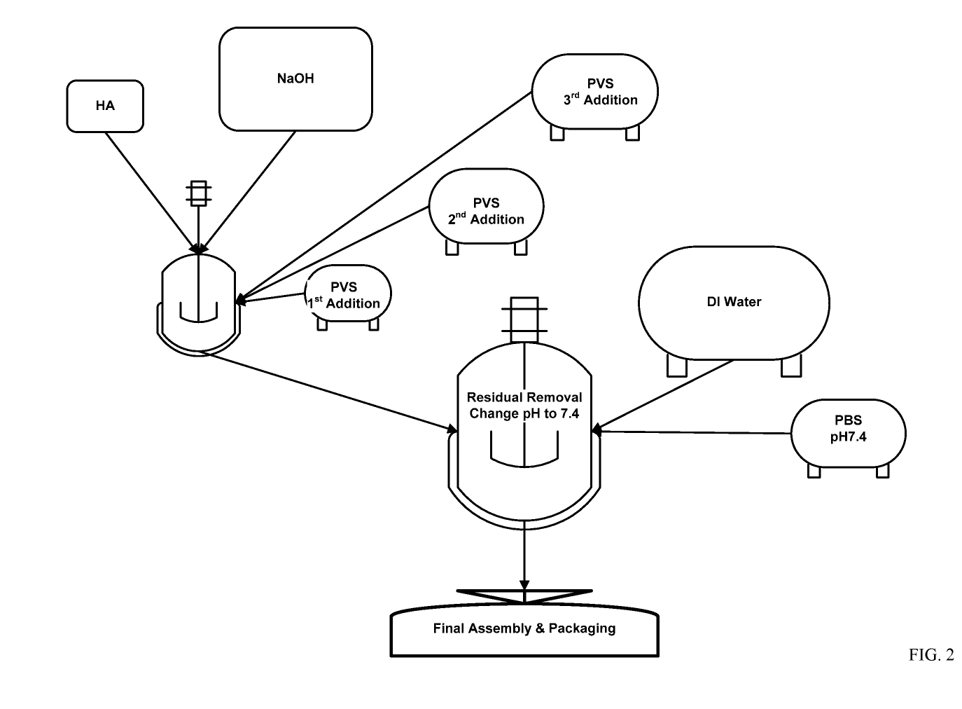

At regular intervals (every 2 hours), the hydrogels were removed during the heat-treatment and decanted, and pH was measured (see FIG. 2). Fresh swelling buffer was used after each measurement. The results show that, for all hydrogels, pH ranged between 11 and 12 after 2-hours of swelling. Then pH gradually decreased to 7.2-7.5.

The decrease was faster for the hydrogels that were less cross-linked, i.e., where the HA/DVS-ratio was higher. The decrease in pH is shown for the HA 6% solution and two different ratios of HA/DVS in FIG. 2, where the HA/DVS ratio of 10:1 is labelled with triangles, and 15:1 is labelled with squares. In these two cases, pH was neutralized within 8 hours. In contrast, neutral pH was reached after 14 hour-swelling for hydrogels with either a higher HA concentration (e.g. 8%) or a higher degree of cross-linking (e.g. HA/DVS ratio of 2.5). These observations are in accordance with the fact that HA molecules in the low cross-linked hydrogels exhibit greater freedom and flexibility, allowing good hydration and thereby faster pH equilibration.

### Example 17

### Visco Elastic Properties of Hydrogels Based on DVS-Crosslinked HA

The rheological measurements were performed on a Physica MCR 301 rheometer (Anton Paar, Ostfildern, Germany) using a plate-plate geometry and at a controlled temperature of 25° C. The visco-elastic behavior of the samples was investigated by dynamic amplitude shear oscillatory tests, in which the material was subjected to a sinusoidal shear strain. First, strain/amplitude sweep experiments were performed to evaluate the region of deformation in which the linear viscoelasticity is valid. The strain typically ranged from 0.01 to 200% and the frequency was set to 1 Hz. Then, in the linear visco-elastic regions, the shear storage modulus (or elastic modulus G′) and the shear loss modulus (or viscous modulus, G″) values were recorded from frequency sweep experiments at a constant shear strain (10%) and at a frequency between 0.1 and 10 Hz. The geometry, the NF and the gap were PP 25, 2 and 1 mm, respectively. G′ gives information about the elasticity or the energy stored in the material during deformation, whereas G″ describes the viscous character or the energy dissipated as heat. In particular, the elastic modulus gives information about the capability of the sample to sustain load and return in the initial configuration after an imposed stress or deformation. In all experiments, each sample was measured at least three times.

In case of the hydrogel with a higher degree of cross-linking (i.e. lower HA/DVS ratio: 10/1) G′ is one order of magnitude higher than G″, indicating that this sample behaves as a strong gel material. Briefly, the overall rheological response is due to the contributions of physical and chemical crosslinks, and to topological interactions among the HA macromolecules. The interactions among the chains bring about a reduction of their intrinsic mobility that is not able to release stress, and consequently the material behaves as a three-dimensional network, where the principal mode of accommodation of the applied stress is by network deformation. Moreover, this hydrogel was more elastic than that with a lower degree of cross-linking (i.e., higher ratio of HA/DVS: 15:1). Indeed, the higher the number of permanent covalent cross-links, the larger the number of entanglements, and therefore the higher the elastic response of the hydrogel.

### Example 18

### Crosslinked HA/DVS Hydrogel with Preservative

A DVS-cross-linked HA hydrogel was prepared using 1.5 g of sodium HA in 0.2 M NaOH to give a 6% (w/v) solution. The HA/DVS weight ratio was 10:1. The hydrogel was prepared in three replicates according to the procedure described in example 2 until the swelling step, after which it was treated as follows: After incubation in an oven at 50° C. for two hours, the hydrogel was immersed into Na2HPO4/NaH2PO4 buffer (1 L, 50 mM, pH 7.0) containing the preservative (2-phenoxyethanol/3[(2-ethylhexyl)oxy]1,2-propanediol).

The concentration of preservative was 10 mL/mL to target a final concentration of 1% (v/v) in the swollen hydrogel. It was anticipated that the preservative would diffuse into the hydrogel during the incubation, and that at the same time, microbial contamination in the buffer would be prevented.

The vessel was covered with parafilm and placed in an oven at 37° C. After 1 h, the swelling bath was removed and the hydrogel was swollen in a fresh phosphate buffer containing 10 mL/mL preservative for 6-7 h. This step was repeated until the swelling time was 12 h, whereafter the pH was measured. Swelling was continued for another 2.5 h to reach neutral pH.

The amount of preservative incorporated into the hydrogel was determined by UV-spectrophotometry (Thermo Electron, Nicolet, Evolution 900, equipment nr. 246-90). A 1% (v/v) solution of the preservative in phosphate buffer was first analyzed to select the wavelength. Approximately 5 mL of hydrogel were collected using a pipette. Typically, samples were collected in the center of the swollen round hydrogel, and in the north, east, south, and west “sides” of the round gel.

The samples were then transferred into a cuvette and the absorbance was read at 292 nm. Each sample was read three times and the absorbance was zeroed against a blank DVS-cross-linked HA hydrogel, containing no preservative.

The results showed that the amount of preservative incorporated in the DVS-HA hydrogel ranged between 0.91% and 1.02% (see Table 10). There was very good reproducibility between the replicates. Importantly, no significant difference between samples from the same hydrogel was observed, indicating a homogenous diffusion of the preservative into the hydrogel.

TABLE 8
Amount of incorporated preservative into
DVS-HA hydrogel upon
swelling in a 1% preservative-spiked
phosphate buffer for 14.5 h.

| | | | Preservative | Average |
|---|---|---|---|---|
| | Sample | Absorbance* | concentration | concentration |
| Sample ID | site | (292 nm) | (%, v/v) | (%, v/v) |
| Replicate 1 | Center | 0.072 | 1.02 | 0.91 |
| | Side | 0.058 | 0.82 | |
| | Side | 0.066 | 0.94 | |
| | Side | 0.057 | 0.81 | |
| | Side | 0.068 | 0.97 | |
| Replicate 2 | Middle | 0.076 | 1.08 | 1.02 |
| | Side | 0.069 | 0.98 | |
| | Side | 0.082 | 1.17 | |
| | Side | 0.071 | 1.01 | |
| | Side | 0.062 | 0.88 | |
| Replicate 3 | Middle | 0.083 | 1.18 | 1.02 |
| | Side | 0.074 | 1.05 | |
| | Side | 0.069 | 0.98 | |
| | Side | 0.066 | 0.94 | |
| | Side | 0.068 | 0.97 | |

### Example 19

### Biodegradable Polymer Choices

The time of degradation may be adjusted based on the polymer mixture in Table 1 below. Examples 1 and 2 below are examples of matrix incorporation of drug or drugs into a biodegradable polymer to control the releases the drugs.

TABLE 1
Biodegradation Time and Composition

| | Degradation Time |
|---|---|
| Polymer | (mos) |
| 50:50 DL-PLG | 1-2 |
| 65:35 DL-PLG | 3-4 |
| 75:25 DL-PLG | 4-5 |
| 85:15 DL-PLG | 5-6 |
| DL-PLA | 12-16 |
| L-PLA | >24 |
| PGA | 6-12 |
| PCL | >24 |

Different types of biodegradable polymer may be used to control the degradation timing and/or to control the degradation by-products. Some biodegradable polymers are:
PGA, PLA and their copolymers are some of the most frequently used biodegradable polymer materials in part because their properties that can be tuned by changing the polymer composition within the basic PLA/PGA theme.
Poly(glycolic acid) (PGA) is very susceptible to hydrolysis
Poly(lactic acid) (PLA) exists in D and/or L enantiomer mixtures of these results in varying biodegradation timing due to crystalline regions that form when they are in mixture which limits the level of hydrolysis possible
Polydioxanone (PDS)
Poly(ε-caprolactone)
Poly(DL-lactide-co-ε-caprolactone)

### Surfactant Choices:

The particle sizes of the micro capsules are directly controlled by the interfacial chemistry of the organic phase and the aqueous phase. A surfactant is often used to mediate interfacial surface chemistry between an oily substance and the aqueous environment. A surfactant is a detergent that is in an aqueous solution. Surfactants are large molecules that have both polar and non-polar ends. The polar end of the molecule will attach itself to water, also a polar molecule. The non-polar end of the molecule will attract NAPL (non-aqueous phase liquid) compounds.

Examples of surfactants that are used for solubilization are:

1. Sioponic 25-9 which is a linear alcohol ethoxylate, and has a solubilization value of 2.75 g/g
2. Tergitol which is an ethylene oxide/propylene oxide with a solubilization value of 1.21 g/g
3. Tergitol XL-80N which is an ethylene oxide propylene oxide alkoxylate of primary alcohol with a solubilization value of 1.022 g/g
4. Tergitol N-10 which is an a trimethyl nonal ethoxylate with a solubilization value of 0.964 g/g
5. Rexophos 25/97 which is a phosphated nonylphenol ethooxylate with a solubilization value of 0.951 g/g

### Example 20

### Biodegradable Micro Particles Containing Anti-Inflammatory, Cortical Steroid or Steroids

a. Delayed 30 days
b. Controlled release over 120 days

### Organic Phase:

Make a 20% DLPLG polymer with methylene chloride
The DLPLA polymer contains 65% DL and 35% PLG
Weigh 0.02 g triamcinolone into a glass vial
Dispense 2 mL of 20% DLPLG polymer solution into the vial containing the triamcinolone
Dissolve the drug completely using an orbital mixer

### Aqueous Phase:

Make 100 mL of SDS (sodium dodecyl sulfate) at a 0.1 molar concentration in DI water
Dispense 8 mL of SDS 0.1 molar solution into the drug/polymer solution

### Solvent Evaporation:

Place the glass vial containing the reaction mixture under the impeller mixer.
Turn the mixer up to 1200 rpm.
Unless the speed required to produce a desired particle size is known, start slowly and work up to an impeller speed that produces the desired particle size.
After the speed to produce the desired particle size has been figured out. Begin heating the vessel in a 80 C water bath with continuous mixing
When all the methylene chloride in the organic phase has been boiled off, this case, the time is 45 minutes, stop heating
Continue mixing, let reaction cool to room temperature slowly
The rate of cooling and mixing effect the agglomeration of the particles to each other
The SDS may be washed by continuously exchanging the solution mixture with DI water
Collect the particles by filtration
Dry the particles at 80 C in a vacuum oven

### Fluidized Bed Encapsulation

Make a 3% and 5% polymer composition 50:50 PL:PLG in methylene chloride
Put the dried particle containing drug into the fluidized bed
Deposit a uniform layer of polymer onto the drug containing particles using the 5% polymer solution. Adjust the spray rate and air flow to get an optimized particle bed.
Use the 3% polymer solution to finalized the process ensuring that there are no pin holes to eventual cause unwanted early release of the drug

### Example 21

### Biodegradable Microcapsule Containing Anti-proliferative Pharmaceutical

a. Delayed 60 days
b. Controlled release over 365 days

### Organic Phase:

Make a 20% DLPLG polymer with methylene chloride
The DLPLA polymer contains 100% PGA
Weigh 0.02 g sirolimus into a glass vial
Dispense 2 mL of 20% DLPLG polymer solution into the vial containing the triamcinolone
Dissolve the drug completely using an orbital mixer

### Aqueous Phase:

Make 100 mL of SDS (sodium dodecyl sulfate) at a 0.1 molar concentration in DI water
Dispense 8 mL of SDS 0.1 molar solution into the drug/polymer solution

### Solvent Evaporation:

Place the glass vial containing the reaction mixture under the impeller mixer.
Turn the mixer up to 1200 rpm.
Unless the speed required to produce a desired particle size is known, start slowly and work up to an impeller speed that produces the desired particle size.
After the speed to produce the desired particle size has been figured out. Begin heating the vessel in a 80 C water bath with continuous mixing
When all the methylene chloride in the organic phase has been boiled off, this case, the time is 45 minutes, stop heating
Continue mixing, let reaction cool to room temperature slowly
The rate of cooling and mixing effect the agglomeration of the particles to each other
The SDS may be washed by continuously exchanging the solution mixture with DI water
Collect the particles by filtration
Dry the particles at 80 C in a vacuum oven

### Fluidized Bed Encapsulation

Make a 3% and 5% polymer composition 65:35 PL:PLG in methylene chloride
Put the dried particle containing drug into the fluidized bed
Deposit a uniform layer of polymer onto the drug containing particles using the 5% polymer solution. Adjust the spray rate and air flow to get an optimized particle bed.
Use the 3% polymer solution to finalized the process ensuring that there are no pin holes to eventual cause unwanted early release of the drug

### Example 22

### Dermal Filler Composition Containing anesthetic, Cortical Steroid and Anti-proliferative Pharmaceutical

a. Biodegradable microcapsule containing a cortical steroid delayed 30 days, controlled release over 120 days
b. Biodegradable microcapsule containing an anti-proliferative pharmaceutical delayed 60 days, controlled released over 365 days

### Composition Mixture (Dry)

| | |
|---|---|
| Hyaluronic acid, cross-linked | 60%-95% |
| Anti-inflammatory drug containing micro particles | 5%-20% |
| Antiproliferative drug containing micro particles | 5%-20% |
| Anesthetic drug (lidocaine hydrochloride) | 0.1%-5% |

Reconstitute in phosphate buffered saline at 0.024 g/mL concentration

### Example 23

### Encapsulation of an Anti-Proliferative Pharmaceutical a Biodegradable Acrylic Acid Copolymer

### Shell Formation Phase

Dissolve the following, which makes up the organic phase:
0.25 g of a biodegradable acrylic acid copolymer in
0.75 g of sirolimus
2 mL methylene chloride
0.1 mL ethanol
Aqueous phase is:
75 mL of 0.5% polyvinyl alcohol solution maintained at room temperature
Disperse the two phases using a mechanical mixer at 1200 rpm or whichever speed that gives the desire particle size
Add an appropriate amount of amine or in this case triethyl amine
Continue mixing for 2 hours with reaction vessel in a water bath at 80 C
Add 00.1 mL of Jeffamine (T-403) to harden the capsule surface
Continue mixing, let reaction cool to room temperature slowly
The rate of cooling and mixing effect the agglomeration of the particles to each other
The polyvinyl alcohol may be washed by continuously exchanging the solution mixture with fresh DI water
Collect the particles by filtration
Dry the particles at 80 C in a vacuum oven

### Fluidized Bed Encapsulation

Make a 3% and 5% polymer composition 65:35 PL:PLG in methylene chloride
Put the dried particle containing drug into the fluidized bed
Deposit a uniform layer of polymer onto the drug containing particles using the 5% polymer solution. Adjust the spray rate and air flow to get an optimized particle bed.
Use the 3% polymer solution to finalized the process ensuring that there are no pin holes to eventual cause unwanted early release of the drug In addition to biocompatibility, the other important characteristics of the gel slurries according to the one embodiment which determine their usefulness in various medical fields is the complex combination of their rheological properties. These properties include viscosity and its dependence on shear rate, the ratio between elastic and viscous properties in dynamic mode, relaxation behavior and some others which are discussed below in more detail. In general, the rheology of the products of the one embodiment can be controlled over very broad limits, essentially by two methods. According to the first such method, the rheological properties of each of the two phases forming the viscoelastic gel slurry are controlled in such a way that gives the desirable rheology for the final product. The second such method of controlling the rheology of the gel slurry consists of selecting a proper ratio for two phases. But because these parameters, i.e. rheology of the two phases and their ratio determine some other important properties of the products of one embodiment, the best way to control the rheology should be selected ad hoc for each specific case.

The gels suitable for the use in the products according to the one embodiment can represent very many different kinds of rheological bodies varying from hard fragile gels to very soft deformable fluid-like gels. Usually, for the gels which are formed without a crosslinking reaction, for example, a conventional gelatin gel, the hardness and elasticity of the gel increases with increasing polymer concentration. The rheological properties of a crosslinked gel are usually a function of several parameters such as crosslinking density, polymer concentration in the gel, composition of the solvent in which the crosslinked polymer is swollen. Gels with different rheological properties based on hyaluronan and hylan are described in the above noted U.S. Pat. Nos. 4,605,691, 4,582,865 and 4,713,448. According to these patents, the rheological properties of the gel can be controlled, mainly, by changing the polymer concentration in the starting reaction mixture and the ratio of the polymer and the crosslinking agent, vinyl sulfone. These two parameters determine the equilibrium swelling ratio of the resulting gel and, hence, the polymer concentration in the final product and its rheological properties.

A substantial amount of solvent can be removed from a gel which had previously been allowed to swell to equilibrium, by mechanical compression of the gel. The compression can be achieved by applying pressure to the gel in a closed vessel with a screen which is permeable to the solvent and impermeable to the gel. The pressure can be applied to the gel directly by means of any suitable device or through a gas layer, conveniently through the air. The other way of compressing the gel is by applying centrifugal force to the gel in a vessel which has at its bottom the above mentioned semipermeable membrane. The compressibility of a polymeric gel slurry depends on many factors among which are the chemical nature of the gel, size of the gel particles, polymer concentration and the presence of a free solvent in the gel slurry. In general, when a gel slurry is subjected to pressure the removal of any free solvent present in the slurry proceeds fast and is followed by a much slower removal of the solvent from the gel particles. The kinetics of solvent removal from a gel slurry depends on such parameters as pressure, temperature, configuration of the apparatus, size of the gel particles, and starting polymer concentration in the gel. Usually, an increase in pressure, temperature, and filtering surface area and a decrease in the gel particle size and the initial polymer concentration results in an increase in the rate of solvent removal.

Partial removal of the solvent from a gel slurry makes the slurry more coherent and substantially changes the rheological properties of the slurry. The magnitude of the changes strongly depends on the degree of compression, hereinafter defined as the ratio of the initial volume of the slurry to the volume of the compressed material.

The achievable degree of compression, i.e. compressibility of a gel slurry, is different for different gels. For hylan gel slurries in saline, for example, it is easy to have a degree of compression of 20 and higher.

Reconstitution of the compressed gel with the same solvent to the original polymer concentration produces a gel identical to the original one. This has been proven by measuring the rheological properties and by the kinetics of solvent removal from the gel by centrifuging.

It should be understood that the polymer concentration in the gel phase of the viscoelastic mixtures according to the one embodiment may vary over broad ranges depending on the desired properties of the mixtures which, in turn, are determined by the final use of the mixture. In general, however, the polymer concentration in the gel phase can be from 0.01 to 30%, preferably, from 0.05 to 20%. In the case of hylan and hyaluronan pure or mixed gels, the polymer concentration in the gel is preferably, in the range of 0.1 to 10%, and more preferably, from 0.15 to 5% when the swelling solvent is physiological saline solution (0.15M aqueous sodium chloride).

As mentioned above the choice of a soluble polymer or polymers for the second phase of the viscoelastic gel slurries according to one embodiment is governed by many considerations determined by the final use of the product. The polymer concentration in the soluble polymer phase may vary over broad limits depending on the desired properties of the final mixture and the properties of the gel phase. If the rheological properties of the viscoelastic gel slurry are of prime concern then the concentration of the soluble polymer may be chosen accordingly with due account taken of the chemical nature of the polymer, or polymers, and its molecular weight. In general, the polymer concentration in the soluble phase may be from 0.01% to 70%, preferably from 0.02 to 40%. In the case when hylan or hyaluronan are used as the soluble polymers, their concentration may be in the range of 0.01 to 10%, preferably 0.02 to 5%. In the case where other glycosaminoglycans such as chondroitin sulfate, dermatan sulfate, etc., are used as the soluble polymers, their concentration can be substantially higher because they have a much lower molecular weight.

The two phases forming the viscoelastic gel slurries according to one embodiment can be mixed together by any conventional means such as any type of stirrer or mixer. The mixing should be long enough in order to achieve uniform distribution of the gel phase in the polymer solution. As mentioned above, the gel phase may already be a slurry obtained by disintegrating a gel by any conventional means such as pushing it through a mesh or a plate with openings under pressure, or by stirring at high speed with any suitable stirrer. Alternatively, the viscoelastic mixed gel slurries can be prepared by mixing large pieces of gel with the polymer solution and subsequently disintegrating the mixture with formation of the viscoelastic slurry by any conventional means discussed above. When the first method of preparing a mixed gel slurry according to one embodiment is used, the gel slurry phase can be made of a gel swollen to equilibrium, and in this case there is no free solvent between the gel particles, or it may have some free solvent between gel particles. In the latter case this free solvent will dilute the polymer solution used as the second phase. The third type of gel slurry used as the gel phase in the mixture is a compressed gel whose properties were discussed above. When a compressed gel slurry is mixed with a polymer solution in some cases the solvent from the solution phase will go into the gel phase and cause additional swelling of the gel phase to equilibrium when the thermodynamics of the components and their mixture allows this to occur.

The composition of the viscoelastic mixed gel slurries according to one embodiment can vary within broad limits. The polymer solution in the mixture can constitute from 0.1 to 99.5%, preferably, from 0.5 to 99%, more preferably, from 1 to 95%, the rest being the gel phase. The choice of the proper composition of the mixture depends on the properties and composition of the two components and is governed by the desirable properties of the slurry and its final use.

The viscoelastic gel mixtures according to one embodiment, in addition to the two major components, namely, the polymeric gel slurry and the polymer solution, may contain many other components such as various physiologically active substances, including drugs, fillers such as microcrystalline cellulose, metallic powders, insoluble inorganic salts, dyes, surface active substances, oils, viscosity modifiers, stabilizers, etc., all depending upon the ultimate use of the products.

The viscoelastic gel slurries according to one embodiment represent, essentially, a continuous polymer solution matrix in which discrete viscoelastic gel particles of regular or irregular shape are uniformly distributed and behave rheologically as fluids, in other words, they exhibit certain viscosity, elasticity and plasticity. By varying the compositional parameters of the slurry, namely the polymer concentration in the gel and the solution phases, and the ratio between two phases, one may conveniently control the rheological properties of the slurry such as the viscosity at a steady flow, elasticity in dynamic mode, relaxation properties, ratio between viscous and elastic behavior, etc.

The other group of properties which are strongly affected by the compositional parameters of the viscoelastic gel slurries according to one embodiment relates to diffusion of various substances into the slurry and from the slurry into the surrounding environment. The diffusion processes are of great importance for some specific applications of the viscoelastic gel slurries in the medical field such as prevention of adhesion formation between tissues and drug delivery as is discussed below in more detail.

It is well known that adhesion formation between tissues is one of the most common and extremely undesirable complications after almost any kind of surgery. The mechanism of adhesion formation normally involves the formation of a fibrin clot which eventually transforms into scar tissue connecting two different tissues which normally should be separated. The adhesion causes numerous undesirable symptoms such as discomfort or pain, and may in certain cases create a life threatening situation. Quite often the adhesion formation requires another operation just to eliminate the adhesions, though there is no guarantee against the adhesion formation after re-operation. One means of eliminating adhesion is to separate the tissues affected during surgery with some material which prevents diffusion of fibrinogen into the space between the tissues thus eliminating the formation of continuous fibrin clots in the space. A biocompatible viscoelastic gel slurry can be successfully used as an adhesion preventing material. However, the diffusion of low and high molecular weight substances in the case of plain gel slurries can easily occur between gel particles especially when the slurry mixes with body fluids and gel particles are separated from each other. On the other hand, when a viscoelastic mixed gel slurry according to one embodiment, is implanted into the body, the polymer solution phase located between gel particles continues to restrict the diffusion even after dilution with body fluids thus preventing adhesion. Moreover, this effect would be more pronounced with an increase in polymer concentration of the polymer solution phase.

The same is true when the viscoelastic mixed gel slurries according to one embodiment are used as drug delivery vehicles. Each of the phases of the slurry or both phases can be loaded with a drug or any other substance having physiological activity which will slowly diffuse from the viscoelastic slurry after its implantation into the body and the diffusion rate can be conveniently controlled by changing the compositional parameters of the slurries.

Components of the viscoelastic mixed gel slurries according to one embodiment affect the behavior of living cells by slowing down their movement through the media and preventing their adhesion to various surfaces. The degree of manifestation of these effects depends strongly on such factors as the composition of the two components of the mixture and their ratio, the nature of the surface and its interaction with the viscoelastic gel slurry, type of the cells, etc. But in any case this property of the viscoelastic gel slurries can be used for treatment of medical disorders where regulation of cell movement and attachment are of prime importance in cases such as cancer proliferation and metastasis.

In addition to the above two applications of biocompatible viscoelastic gel slurries according to one embodiment other possible applications include soft tissue augmentation, use of the material as a viscosurgical tool in opthalmology, otolaryngology and other fields, wound management, in orthopedics for the treatment of osteoarthritis, etc. In all of these applications the following basic properties of the mixed gel slurries are utilized: biocompatibility, controlled viscoelasticity and diffusion characteristics, easily controlled residence time at the site of implantation, and easy handling of the material allowing, for example its injection through a small diameter needle. The following methods were used for characterization of the products obtained according to one embodiment. The concentration of hylan or hyaluronan in solution was determined by hexuronic acid assay using the automated carbazole method (E. A. Balazs, et al, Analyt. Biochem. 12, 547-558, 1965). The concentration of hylan or hyaluronan in the gel phase was determined by a modified hexuronic acid assay as described in Example 1 of U.S. Pat. No. 4,582,865.

Rheological properties were evaluated with the Bohlin Rheometer System which is a computerized rheometer with controlled shear rate and which can operate in three modes: viscometry, oscillation and relaxation. The measurements of shear viscosity at low and high shear rates characterize viscous properties of the viscoelastic gel slurries and their pseudoplasticity (the ratio of viscosities at different shear rates) which is important for many applications of the products. Measurements of viscoelastic properties at various frequencies characterized the balance between elastic (storage modulus G′) and viscous (loss modulus G″) properties. The relaxation characteristics were evaluated as the change of the shear modulus G with time and expressed as the ratio of two modulus values at different relaxation times.

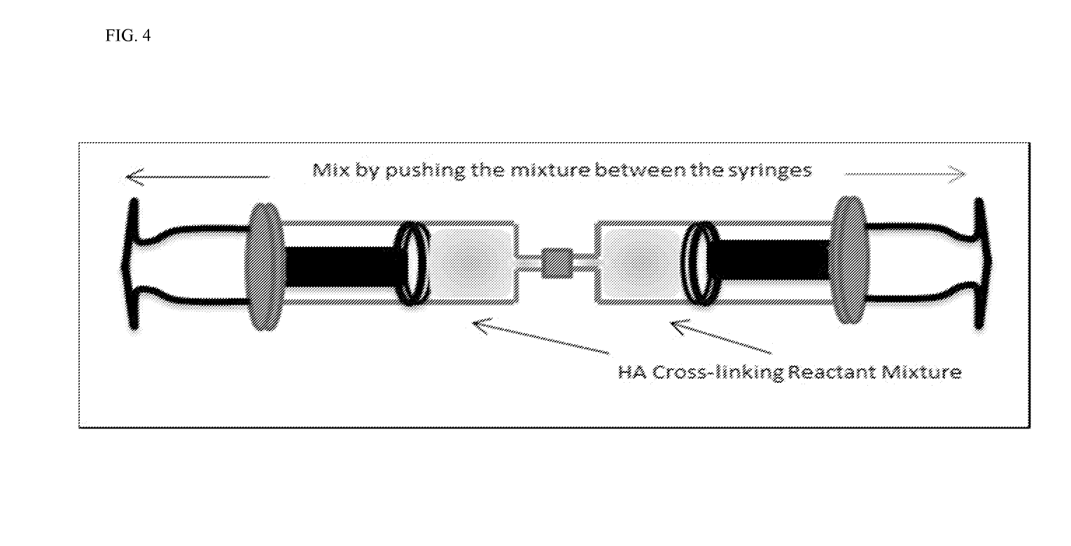

Next, various HA Crosslinking Approaches are discussed. The following reactions focus mainly on the two most reactive functional groups—the hydroxyl and the carboxyl.
1. Bisepoxide,
   Ethyleneglycol diglycidyl ether
   1,4-butanediol diglycidyl ether
   This method was originally developed to crosslink agarose. Currently to crosslink HA the reaction is in dilute NaOH using bisepoxybutane and sodium borohydride. Reaction of hyaluronan with ethyleneglycol diglycidyl ether in ethanolic 0.1 N NaOH at 60° C. also afforded a hydrogel (FIG. 4A). The resulting gels had high water contents (>95%) and were investigated for use as an inflammation (stimulus)-responsive degradable matrix for implantable drug delivery. A hydrogel prepared from hyaluronan and alkaline 1,4-butanediol diglycidyl ether was highly porous. This material was then activated with perioxidate and then modified with an 18-amino acid peptide containing a cell attachment domain, Arg-Gly-Asp (RGD), to enhance cell attachment to the hydrogel. In alkaline medium, divinyl sulfone also cross-links hyaluronan, most likely via reaction with hydroxyl groups.

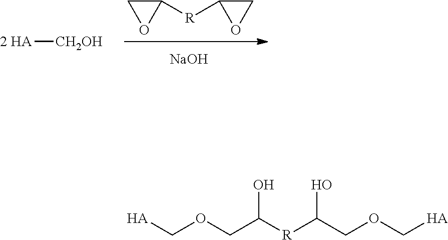

2. Divinylsulfone (DVS)
   In alkaline medium, divinyl sulfone also cross-links hyaluronan, most likely via reaction with hydroxyl groups.

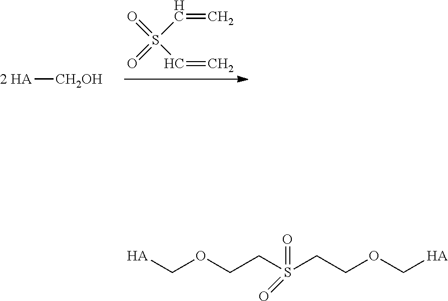

3. Internal esterification
   The autocross-linked polymer (ACP™, Fidia) is an internally esterified derivative of hyaluronan, with both inter- and intra-molecular bonds between the hydroxyl and carboxyl groups of hyaluronan. ACP™ can be lyophilized to a white powder and hydrated to a transparent gel. This novel biomaterial has been used as a barrier to reduce post-operative
4. Photo-cross Linking
   A methacrylate derivative of hyaluronan was synthesized by the esterification of the hydroxyls with excess methacrylic anhydride, as described above for hyaluronan butyrate. This derivative was photocross-linked to form a stable hydrogel using ethyl eosin in 1-vinyl-2-pyrrolidone and triethanolamine as an initiator under argon ion laser irradiation at 514 nm. The use of in situ photopolymerization of an hyaluronan derivative, which results in the formation of a cohesive gel enveloping the injured tissue, may provide isolation from surrounding organs and thus prevent the formation of adhesions. A preliminary cell encapsulation study was successfully performed with islets of Langerhans to develop a bioartificial source of insulin.
5. Glutaraldehyde cross linking
   Hyaluronan strands extruded from cation-exchanged sodium hyaluronate (1.6 MDa) were cross-linked in glutaraldehyde aqueous solution, although the chemical nature of this process was not identified. The strand surfaces were then remodeled by attachment of poly-D- and poly-L-lysine. The polypeptide-resurfaced hyaluronan strands showed good biocompatibility and promoted cellular adhesion.
6. Metal cation mediated cross linking
   Intergel® (FeHA, LifeCore) is a hydrogel formulation of hyaluronan formed by chelation with ferric hydroxide. Similar cross-linking of yaluronan has been the basis of preparations using copper, zinc, calcium, barium, and other chelating metals. The reddish FeHA gel is in development for prevention of post-surgical adhesions.
7. Carbodiimide cross linking
   Incert® is a bioresorbable sponge (Anika Therapeutics) prepared by cross-linking hyaluronan with a biscarbodiimide in aqueous isopropanol. This procedure takes advantage of the otherwise undesirable propensity of carbodiimides to react with hyaluronan to form N-acylureas. In this application, the formation of two N-acylurea linkages provides a chemically stable and by-product-free cross-link. Because of the hydrophobic biscarbodiimides employed, Incert® adheres to tissues without the need for sutures and retains its efficacy even in the presence of blood. Recently, it was found to be effective at preventing post-operative adhesions in a rabbit fecal abrasion study.

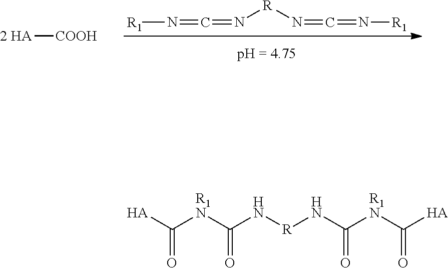

A low-water content hyaluronan hydrogel film was made by cross-linking a hyaluronan (1.6 MDa) film with a water-soluble carbodiimide as a coupling agent in an aqueous mixture containing a water-miscible non-solvent of hyaluronan. The highest degree of cross-linking that gave a low-water content hydrogel was achieved in 80% ethanol. This film, having 60% water content, remained stable for two weeks after immersion in buffered solution. The cross-linking of hyaluronan films with a water-soluble carbodiimide in the presence of L-lysine methyl ester further prolonged the in vivo degradation of a hyaluronan film.
8. Hydrazide cross linking
   Using the hydrazide chemistry described above, hydrogels have been prepared using bishydrazide, trishydrazide, and polyvalent hydrazide compounds as cross-linkers. By adjusting the reaction conditions and the molar ratios of the reagents, gels with physicochemical properties ranging from soft-pourable gels to more mechanically-rigid and brittle gels could be obtained. HA-ADH can be cross-linked using commercially-available small molecule homobifunctional cross-linkers
   More recently, an in situ polymerization technique was developed by cross-linking HA-ADH with a macromolecular cross-linker, PEG-dialdehyde under physiological conditions.
   Biocompatible and biodegradable hyaluronan hydrogel films with well-defined mechanical strength were obtained after the evaporation of solvent. Macromolecular drugs were released slowly from these hyaluronan hydrogel films, and these new materials accelerated re-epithelialization during wound healing.

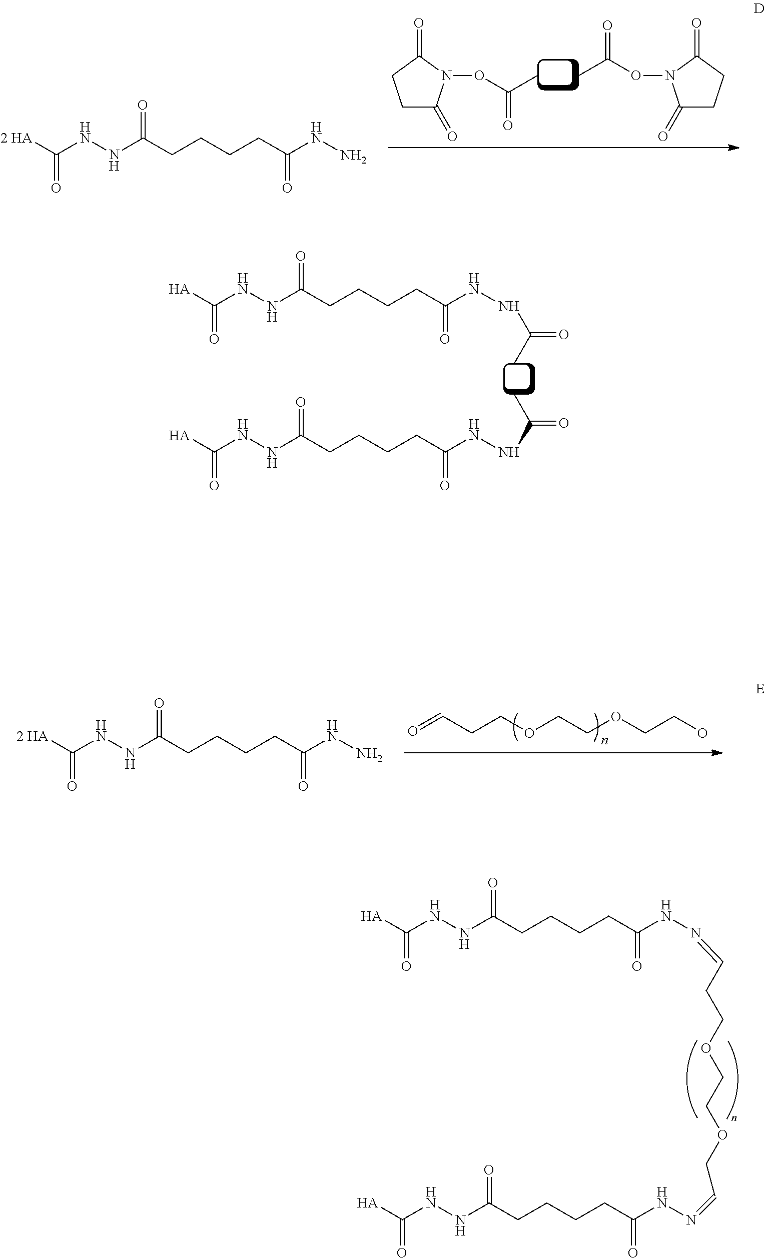

1. Cross linking with residual proteins
   Example of this is Hylans (Biomatrix) are hydrogels or hydrosols formed by cross-linking hyaluronan-containing residual protein with formaldehyde in a basic solution. 13 Soluble hylan is a high molecular weight form (8-23 MDa) of hyaluronan that exhibits enhanced rheological properties compared to hyaluronan. Hylan gels have greater elasticity and viscosity than soluble hylan materials, while still retaining the high biocompatibility of native hyaluronan. Hylans have been investigated in a number of medical applications.
2. Multi-component reactions
   These are 3 to 4 component reactions known as (1) the Passerini reaction and (2) Ugi reactions.
   In the Passerini reaction, an aqueous solution of hyaluronan is mixed with aqueous glutaraldehyde (or another water-soluble dialdehyde) and added to a known amount of a highly reactive isocyanide, e.g., cyclohexylisocyanide.
   In the Ugi four-component reaction (FIG. 4F), a diamine is added to this three-component mixture.
   The degree of cross-linking is controlled by the amount of aldehyde and diamine.

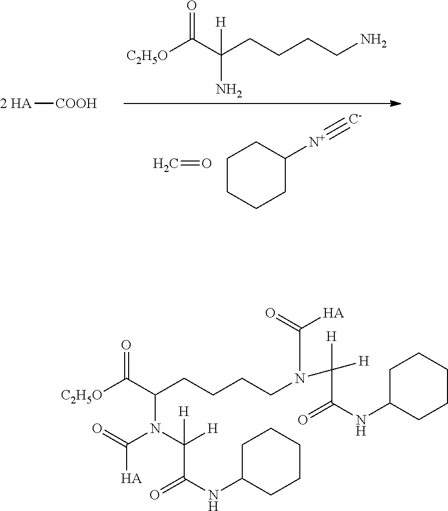

3. Surface modifications
   One example has to do with the Surfaces of polypropylene (PP) and polystyrene (PS) were activated with argon gas and ammonia gas plasmas to emanate the polymer surface. Emanated surfaces were then modified with succinic anhydride to give pendant carboxylic acid groups on the surface, which were then condensed with HA-ADH in the presence of a carbodiimide to give hydrophilic, non-adhesive, and lubricious plastic surfaces. Metal and glass surfaces can also be modified by surface activation followed by covalent chemical attachment of an appropriate hyaluronan derivative.
      2. There are four different therapeutic modification options for HA as shown below
1. A: HA can be cross-linked at two locations: (1) the hydroxyl location and (2) the carboxyl location.
2. B: Drugs that have functional groups that favor reacting with hydroxyl and/or carboxyl could be conjugated on the HA molecule, and the HA molecule will act as a carrier of the drug.
3. C: Individual HA molecules could be grafted or attached covalently to a polymer chain that has pendant function groups which favor reacting with hydroxyl and/or carboxyl.
4. D. HA molecules can be grafted onto a liposome provided that their function groups favor reacting.
   HA Therapeutic Modification Options
      Include cross-linked HA hydrogel, HA drug bioconjugate, HA-grafted copolymers, and HA liposomes
   HA Reactive Sites

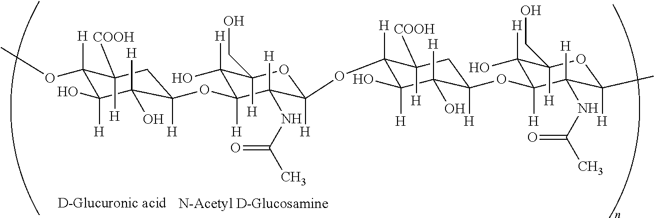

5. Carboxyl group chemical reactions
   1. Esterification

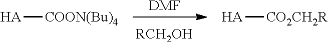

Esterified hyaluronan biomaterials have been prepared by alkylation of the tetra (n-butyl) ammonium salt of hyaluronan with an alkyl halide in dimethylformamide (DMF) solution. These hyaluronan esters can be extruded to produce membranes and fibers, lyophilized to obtain sponges, or processed by spray-drying, extraction, and evaporation to produce microspheres. These polymers show good mechanical strength when dry, but the hydrated materials are less robust. The degree of esterification influences the size of hydrophobic patches, which produces a polymer chain network that is more rigid and stable, and less susceptible to enzymatic degradation.
2. Carbodiimide-mediated reactions

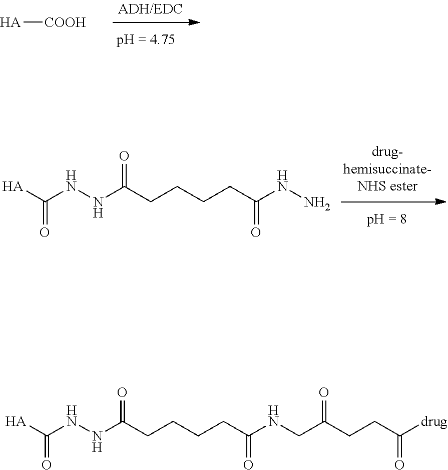

3. The chemical modification of the carboxylic functions of hyaluronan by carbodiimide compounds is generally performed in water at pH 4.75.
6. Hydroxyl group chemical reactions
   1. Sulfation
      The sulfation of hyaluronan with a sulfur trioxide-pyridine complex in DMF produced different degrees of sulfation, HyalSx, where x=1-4 per disaccharide. The sulfated hyaluronic acid HyalS3.5 was then immobilized onto plasma-processed polyethylene (PE) using a diamine polyethylene glycol derivative and a water-soluble carbodiimide. The thrombin time test and platelet adhesion behavior indicated that this procedure was promising for the preparation of blood-compatible, anti-thrombotic PE surfaces. In addition, HyalSx was converted to a photo labile azidophenylamino derivative and was photoimmobilized onto a poly(ethylene terephthalate) (PET) film. 9 Surfaces coated with sulfated hyaluronan exhibited marked reduction of cellular attachment, fouling, and bacterial growth compared with uncoated surfaces, and the coating was stable to degradation by chondroitinase and hyaluronidase.
      Hyaluronan butyrate is used as targeted drug-delivery system specifically to tumor cells. Butyric acid is known to induce cell differentiation and to inhibit the growth of a variety of human tumors was coupled to hyaluronan via the reaction between butyric anhydride and the sym-collidinium salt of low molecular weight hyaluronan in DMF containing dimethylaminopyridine.
   2. Isourea coupling or cyanogen bromide activation
      The anthracycline antibiotics adriamycin and daunomycin were coupled to hyaluronan via cyanogen bromide (CNBr) activation. This reaction scheme is commonly used to activate oligosaccharides to produce affinity matrices via a highly-reactive isourea intermediate. The therapeutic agents appear to become attached via a urethane bond to one of the hydroxylic functions of the oligosaccharide or the glycosaminoglycan, but no spectroscopic verification was provided. Moreover, the harshness of the reaction conditions may compromise the integrity and biocompatibility of the hyaluronan.

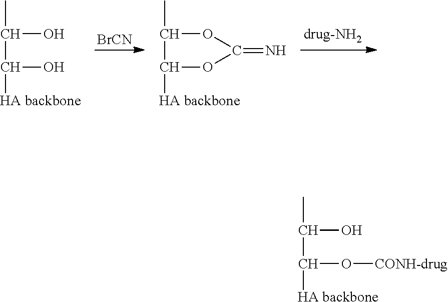

3. Peroxidase oxidation
   Reactive bisaldehyde functionalities can be generated from the vicinal secondary alcohol functions on hyaluronan by oxidation with sodium peroxidase. This chemistry is a standard method for chemical activation of glycoproteins for affinity immobilization or conversion to a fluorescent probe. With peroxidase-activated hyaluronan, reductive coupling with primary amines can give cross-linking, attachment of peptides containing cell attachment domains, or immobilized materials. The harsh oxidative treatment also introduces chain breaks and potentially immunogenic linkages into the hyaluronan biomaterial.

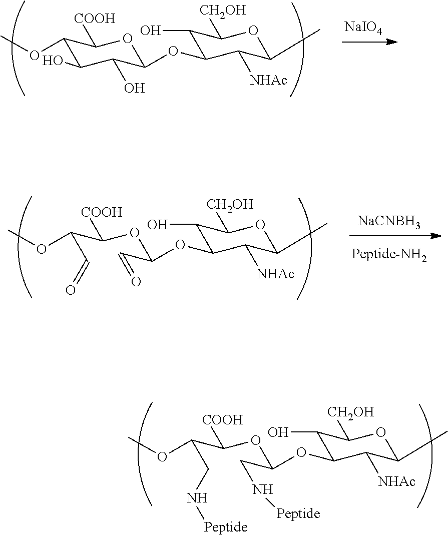

4. Reducing end modification
   Reductive amination of the reducing end of hyaluronan has been employed to prepare affinity matrices, fluorophore-labeled materials, and hyaluronan-phospholipids for insertion into hyaluronan-liposomes. For example, low molecular weight hyaluronan was covalently attached to phosphatidyl-ethanolamine, and this conjugate has been employed for a protective “sugar decoration” on the surface of low density lipoprotein (LDL) particles. End-labeling has not otherwise been extensively used for hyaluronan biomaterials or pro-drug applications, since there is only one attachment point per glycosaminoglycan. This severely limits loading and cross-linking possibilities for high molecular weight hyaluronan.
5. Amide modifications
   Native hyaluronan has, in some preparations, an undetermined number of naturally deacylated glucosamine units that may also be derivatized. As with the reducing end modification, this provides very low modification rates. However, modification of the N-acetyl groups can be important if the commonly used hydrazinolysis method is employed. Limited hydrazinolysis of hyaluronan creates free glucosamine residues on hyaluronan, but can also result in base-induced backbone cleavage and reducing end modification.
      In yet other experiments, the Materials can include

### 1. Experimental Methods

1. Experiment 001-12: Water in oil emulsion cross-linking reaction

Aqueous phase mix

| COMPONENTS | Quantity |
|---|---|
| Hyaluronic acid sodium | 6.5% |
| NaOH | 2M |
| Make total final volume | 0.54 mL |

Oil phase mix

| COMPONENTS | AMOUNT | |
|---|---|---|
| Isooctane | 13 | mL |
| Sodium-bis-sulfosucinate 0.2M | 1 | mL |
| Trimethylpentane 0.04M | 1 | mL |
| DVS | 45 | μL |

1. The reaction is a water in oil emulsion reaction
2. Let it react at RT for 1 hour
3. Collect the gel particles by centrifuge
4. Wash with acetone

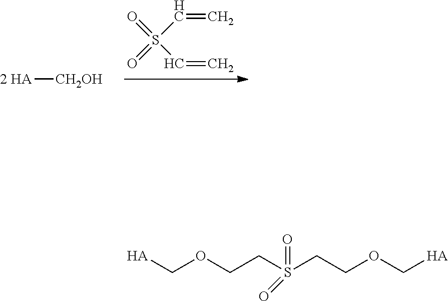

2. Experiment 001-14

Reaction Mixture

| COMPONENTS | AMOUNT |
|---|---|
| Hyaluronic acid | 0.105 g |
| X-Linker Mix: a, b, c, d and e | 0.775 g |

X-Linker Mix

| | AMOUNT | | | | |
|---|---|---|---|---|---|
| COMPONENTS | a | b | c | d | e |
| NaOH 1% | 9.99 | 9.98 | 9.97 | 9.96 | 9.95 |
| BDDE | .010 | .020 | .030 | .040 | .050 |

1. The X-Linker mix is made up first
2. Make up the reaction mixture next
3. Add 0.775 g of the x-linker mix “a” through “e” to the HA. There are reactions.
4. Mix well with a spatula to work the x-linker into the HA
5. Let each reaction take place at RT with mixing every 30-60 min
6. After 8 hours of reacting the product is a cross linked hyaluronic acid gel
7. Placed into a 52 C for 3 hours with mixing every 0.5 hours
8. Washed 3× with PBS

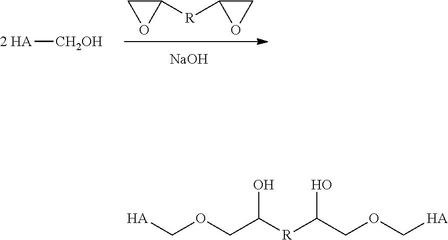

3. Boundary Conditions of Components in the HA X-Linking Process
   Experiment 001-16: X-Linker mix storage life and Reaction Temperature
      1. The X-linker mix must be used sooner than 24 hours after made up and kept at RT conditions
      1. The reaction temperature of 50 C is too high to be kept for more than 1 hour.
   Experiment 001-17: Storage life for 1% NaOH
      2. NaOH solution containing x-linker should be used with 1 hour of its preparation
      3. NaOH concentration of 1 normal is too low to yield completely reacted product
   2. X-Linker Storage Life—BDDE
      1. Experiment 001-18: Showed that once mixed with NaOH, the mixture containing BDDE should be used within 3 hours.
4. X-Linker Storage Life—DVS “TBD”
5. Experiment 001-19

| | |
|---|---|
| COMPONENTS | AMOUNT |

Mixture A

| | | |
|---|---|---|
| Empty culture tube | 8.755 | g |
| HA | 0.105 | g |
| NaOH 1N | 0.5 | mL |

Mixture B

| | | |
|---|---|---|
| NaOH 1N | 2 | mL |
| BDDE | 0.02 | mL |

Final Mixture

| Mixture A | All | |
|---|---|---|
| Mixture B | 0.5 | mL |

1. Mix well after added A and B together
2. Let Stand at RT for 2 hours with mixing every 30 min
3. Let stand in 50 C for 1 hour with mixing every 30 min
4. Product looks very much like commercial product, Juvederm
6. Experiment 001-21

| | |
|---|---|
| COMPONENTS | AMOUNT |

Mixture A

| | | |
|---|---|---|
| Empty culture tube | 10.510 | g |
| HA | 0.105 | g |
| NaOH 1% | 0.5 | mL |

Mixture B

| | |
|---|---|
| NaOH 1% | 9.9 |
| DVS (divinyl sulfone) | .010 |

Final Mixture

| Mixture A | All | |
|---|---|---|
| Mixture B1-B5 | 0.775 | mL |

1. Mix well after added A and B1 through B5 respectively together
2. Let Stand at RT for 2 hours with mixing every 30 min
3. Let stand in 50 C for 1 hour with mixing every 30 min
4. Product looks very much like a commercial product, Juvederm
7. Effects of X-Linking Levels
1. Experiment 001-22: BDDE (1,4-butanediol diglycidylether)

(HA Mix) × 4

| COMPONENTS | AMOUNT | |
|---|---|---|
| HA | 0.105 | g |
| X-Linker Mix_{—} | 0.775 | mL |

BDDE Mix A

| | | |
|---|---|---|
| NaOH 1% | 9.99 | mL |
| BDDE | 0.01 | mL |

BDDE Mix B

| | | |
|---|---|---|
| BDDE Mix A | 1 | mL |
| NaOH 1% | 1 | mL |

BDDE Mix C

| | | |
|---|---|---|
| BDDE Mix A | 1 | mL |
| NaOH 1% | 2 | mL |

BDDE Mix D

| | | |
|---|---|---|
| BDDE Mix A | 1 | mL |
| NaOH 1% | 3 | mL |

2. Experiment 001-25: DVS (Divinyl sulfone)

(HA Mix) × 4

| COMPONENTS | AMOUNT | |
|---|---|---|
| HA | 0.130 | g |
| X-Linker Mix_{—} | 0.800 | mL |

DVS Mix A

| | | |
|---|---|---|
| NaOH 1% | 9.99 | mL |
| DVS (divinyl sulfone) | 0.01 | mL |

DVS Mix B

| | | |
|---|---|---|
| DVS Mix A | 1 | mL |
| NaOH 1% | 1 | mL |

DVS Mix C

| | | |
|---|---|---|
| DVS Mix A | 1 | mL |
| NaOH 1% | 2 | mL |

DVS Mix D

| | | |
|---|---|---|
| DVS Mix A | 1 | mL |
| NaOH 1% | 3 | mL |

In one embodiment, the HA can be serially cross-linked to form a system with monophasic characteristics. The forming a biocompatible cross-linked polymer as an IPN can be done by cross-linking a heteropolysaccharide to form a single cross-linked material; and performing one or more additional cross-linkings on the single cross-linked material to form a multiple cross-linked material, wherein the multiple cross-linked material has a core that lasts longer in a human body than the single cross-linked material. The result is a material with a smooth continuum from slightly cross-linked to the core which is highly cross-linked. The slightly cross-linked material enables the HA to be easily inserted into the human body with a small gauge syringe, but such slightly cross-linked material will not last long in the human body. However, the highly cross-linked material will remain longer in the human body so that the body augmentation does not need periodic touch-ups as is needed by conventional HA dermal fillers.

The cross-link time resulting from the use of a stable, non-aqueous suspension of a delayed cross-linker according to the preferred embodiment may be controlled by varying any one or all of the following:

1) the cross linking compound used,
2) the particle size of the HA in suspension,
3) the pH of the fluid containing the HA,
4) the concentration (i.e., loading) of the HA suspension,
5) the temperature of the solution.
Illustratively, when used under similar conditions, the type of molecular weight of the HA compound may be employed effectively to control the exact cross-linking time of the water-soluble solution. More particularly, suspensions of larger molecular weight HA cross-link more slowly than suspensions of low molecular weight acid.

With respect to the particle size of the suspended halyuronic acid, as particle size increases, the time required for the cross-linking of a water-soluble polymer solution increases. Conversely, as the particle size decreases, the time required for the cross-linking of a water soluble decreases.

The pH of the water soluble polymer solution prior to its cross-linking may be used to control cross-link time. The pH of the water soluble polymer solution affects the solubility rate of the stable, non-aqueous suspension of a delayed cross-linker. Specifically, as the pH of the water soluble polymer solution increases, the solubility rate of the cross-linker suspension increases if the suspension contains a majority of HA particles, whereas the solubility rate of the cross-linker suspension decreases if the suspension contains a majority of borax particles. Conversely, as the pH of the water soluble polymer solution decreases, the solubility rate of the cross-linker suspension decreases if the suspension contains a majority of boric acid particles, whereas the solubility rate of the cross-linker suspension increases if the suspension contains a majority of HA particles.

Both the concentration (i.e., loading) of the stable, non-aqueous suspension of a delayed HA cross-linker in the water soluble polymer solution and the content of the cross-linker suspension affect the cross-link time of a water soluble polymer solution similarly. As either the concentration of the suspension of delayed HA cross-linker in the water-soluble polymer solution or the content of the cross-linker suspension increase, the cross-link time of the water soluble polymer solution decreases. Conversely, as either the concentration of the suspension of the delayed boron cross-linker in the water soluble polymer solution and the content of the cross-linker suspension decrease, the cross-link time of the water soluble polymer solution increases.

Temperature may be used to alter the cross-link time of a water soluble polymer solution. As the temperature of the water soluble polymer solution increases, its cross-link time decreases. Conversely, as the temperature of the water soluble polymer solution decreases, its cross-link time increases. Furthermore, the cross-link time of a water-soluble polymer may be increased or decreased depending upon the clay type utilized in the formulation of the stable, non-aqueous suspension of a delayed HA cross-linker.

In addition, materials such as polymeric microspheres, polymer micelles, soluble polymers and hydrogel-type materials can be used for providing protection for pharmaceuticals against biochemical degradation, and thus have shown great potential for use in biomedical applications, particularly as components of drug delivery devices. The design and engineering of biomedical polymers (e.g., polymers for use under physiological conditions) are generally subject to specific and stringent requirements. In particular, such polymeric materials must be compatible with the biological milieu in which they will be used, which often means that they show certain characteristics of hydrophilicity. They also have to demonstrate adequate biodegradability (i.e., they degrade to low molecular weight species. The polymer fragments are in turn metabolized in the body or excreted, leaving no trace). Biodegradability is typically accomplished by synthesizing or using polymers that have hydrolytically unstable linkages in the backbone. The most common chemical functional groups with this characteristic are esters, anhydrides, orthoesters, and amides. Chemical hydrolysis of the hydrolytically unstable backbone is the prevailing mechanism for the degradation of the polymer. Biodegradable polymers can be either natural or synthetic. Synthetic polymers commonly used in medical applications and biomedical research include polyethyleneglycol (pharmacokinetics and immune response modifier), polyvinyl alcohol (drug carrier), and poly(hydroxypropylmetacrylamide) (drug carrier). In addition, natural polymers are also used in biomedical applications. For instance, dextran, hydroxyethylstarch, albumin and partially hydrolyzed proteins find use in applications ranging from plasma substitute, to radiopharmaceutical to parenteral nutrition. In general, synthetic polymers may offer greater advantages than natural materials in that they can be tailored to give a wider range of properties and more predictable lot-to-lot uniformity than can materials from natural sources.

In one embodiment, the linker is a dicarboxylic acid with at least three atoms between the carbonyls and contains a heteroatom alpha to the carbonyl forming the ester, the release half-life is less than about 10 hours; when Linker is a dicarboxylic acid with at least three atoms between the carbonyls with no heteroatom alpha to the carbonyl forming the ester, the release half-life is more than about 100 hours; wherein when Linker is a dicarboxylic acid with two atoms between the carbonyls and Tether contains a nitrogen with a reactive hydrogen, the release half-life of the HA is from about 0.1 hours to about 20 hours; wherein the release half-life being measured in 0.05M phosphate buffer, 0.9% saline, pH 7.4, at 37° C.; with the proviso that the conjugate is not PHF-SA-Gly-CPT, PHF-(methyl)SA-Gly-CPT, PHF-(2,2-dimethyl)SA-Gly-CPT, PHF-(2-nonen-2-yl)SA-Gly-CPT, PHF-SA-Gly-Taxol, or PHF-SA-Gly-Illudin.

In some embodiments, the polyal is an acetal. In other embodiments, the polyal is a ketal. In some embodiments, the acetal is PHF. In some embodiments, Ri is H. In other embodiments, Ri is CH3. In some embodiments, R2 is —CH(Y)—C(O)—, wherein Y is one of the side chains of the naturally occurring amino acids. In some embodiments, R2 is an aryl group. In some embodiments, R2 is an heteroaryl group. In other embodiments, R2 is an aliphatic ring. In some embodiments, R2 is an aliphatic chain. In some embodiments, R2 is a heterocyclic aliphatic ring. In some embodiments, R1 and R2 when taken together with nitrogen to which they are attached form a ring. Other embodiments are known to those skilled in the art. For example, some embodiments are discussed in US2010/036413, the content of which is incorporated by reference.

FIG. 1 shows an exemplary system to serially produce multiply cross-linked HA. In FIG. 1, HA material P-15 and sodium hydroxide P-16 is provided to a gate and measurement unit P14. The output is provided to a mixer P17. A cross-linker source E9 is provided to a reactor I-7 whose output is stored at a tank P21. The stored cross-linked HA can then be atomized.

FIG. 2 shows another exemplary system to serially produce multiply cross-linked HA. In FIG. 2, HA and sodium hydroxide is provided to a reactor that receives a plurality of cross-linker sources such as PVS1, PVS2, and PVS3 sources. The reactor generated serially and multiply cross-linked HA is then cleaned at a chamber to remove residuals and to change pH to about 7.4. The chamber receives distilled water and PBS at a pH of about 7.4. The cleaned output is then sent to a final assembly and packaging station.

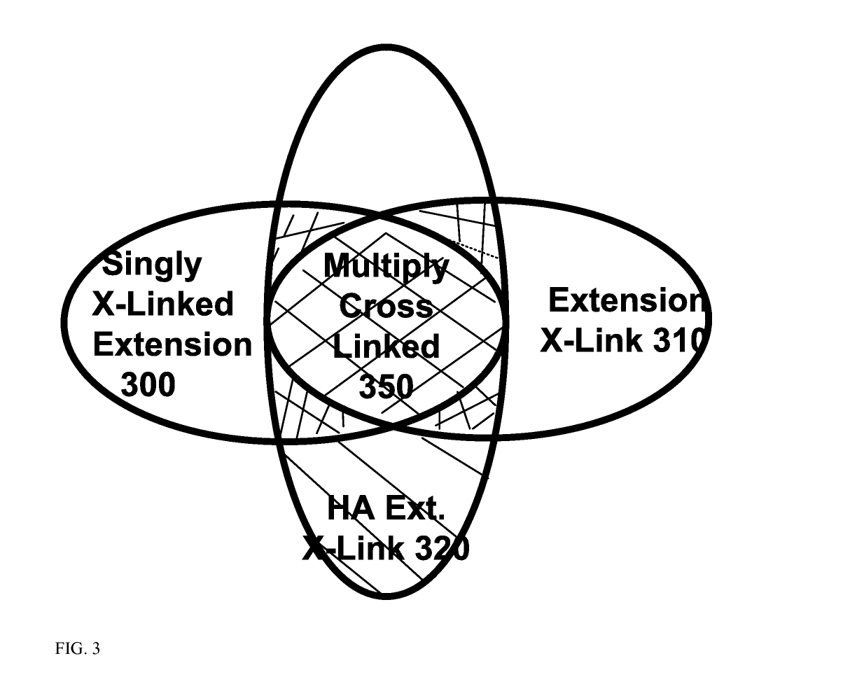

FIG. 3 shows an exemplary diagram of the resulting multiply cross-linked HA. As shown in FIG. 3, the composition includes a first portion 300 of a first polymer with lightly cross-linking extensions or arms; a second portion 310 of polymer with a first serially cross-linked center overlapping the first portion and one or more lightly cross-linked extensions adjacent the serially cross-linked center; and a third portion 320 of polymer with a second serially cross-linked region 350 overlapping the second portion and one or more lightly cross-linked extensions adjacent the serially cross-linked center; wherein the lightly cross-linked extensions enable the composition to be injected through a small gauge needle and the second serially cross-linked center is resistant to absorbtion by biological processes. The region 350 can be multiply cross-linked for biodegradation resistance. The polymer can be one of: collagens, hyaluronic acids, celluloses, proteins, saccharides, an extracellular matrix of a biological system.

In another embodiment, a biocompatible cross-linked IPN polymer can be done by cross-linking a heteropolysaccharide to form a first cross-linked material; and by performing one or more additional cross-linking of the first cross-linked material to form a multiple cross-linked material. The result monophasic HA can be used for augmenting soft tissue with the biocompatible cross-linked polymer.

Besides the foregoing methods of obtaining IPN and semi-IPN by crosslinking both of the components of the blend, semi-IPN can also be obtained by the polymerization of a monomer in the presence of a crosslinking agent and in the presence of the natural acidic polysaccharide or a semisynthetic ester-type derivative thereof.

In the following examples, the HA composition percentage is varied from 75% to 99% of the total composition while the cross linker percentage is varied between 1 and 25% as follows:

| | | | | | | | | | | | |
|---|---|---|---|---|---|---|---|---|---|---|---|
| HA | Composition % | 99 | 90 | 85 | 80 | 75 | 99 | 90 | 85 | 80 | 75 |
| HA | Formula % | 9 | 9 | 8.5 | 8 | 7.5 | 19.8 | 18 | 17 | 16 | 15 |
| 0.5M | Formula % | 90 | 90 | 90 | 90 | 90 | 80 | 80 | 80 | 80 | 80 |
| NaOH | | | | | | | | | | | |
| X-Linker | Formula % | 1 | 1 | 1.5 | 2 | 2.5 | 0.2 | 2 | 3 | 4 | 5 |
| X-Linker | Composition % | 1 | 10 | 15 | 20 | 25 | 1 | 10 | 15 | 20 | 25 |

As the percentage of HA increases, the material is soft, but less resistant to biodegration. As more cross-linker is introduced, the material becomes more hardened and lasts longer. The multiple serially cross-linking processes provide advantages of being soft to the touch, yet long lasting. The varying mechanical/physical properties that constantly becomes softer while remaining tough radiating out from the IPN makes the polymer tough and at the same time compliant with its surrounding for better biocompatibility and feels more natural to the touch. The IPN is an intimate combination of two or more polymer systems, both in network form, at least one of which is synthesized or cross-linked in the immediate presence of the other. If one of the two polymers is in network form (cross-linked) and the other is a linear polymer (not cross-linked), a semi-IPN results. The term IPN currently covers new materials where the at least two polymers in the mixture are not necessarily bound together, but the components are physically associated.

| | | | 1st | 2nd | 3rd | 4th |
|---|---|---|---|---|---|---|
| | Composition % | 99 | | | | |
| HA | Formula % | 9.9 | | | | |
| 0.5M | NaOH | 90 | | | | |
| X-Linker | Formula % | 0.1 | | | | |

| | | | |
|---|---|---|---|
| X-Linker | Composition % | 1 | SERIAL X-L VARIATIONS FOR A COMPOSITION (% of X-Linker in total) |

| XL Type | XL-1, 2, 3 or 4 | XL-1, 2, 3 or 4 | XL-1, 2, 3 or 4 | XL-1, 2, 3 or 4 | |
|---|---|---|---|---|---|
| A | 33.3 | 33.3 | 33.3 | | 100 |
| B | 20 | 30 | 50 | | 100 |
| C | 10 | 20 | 30 | 40 | 100 |
| D | 25 | 25 | 25 | 25 | 100 |
| E | 10 | 20 | 30 | 40 | 100 |

X-LINKER TYPES

| | |
|---|---|
| XL-1 | divinyl sulfone |
| XL-2 | 1,4-butane dioldiglycidyl ether |
| XL-3 | 1,2,3,4,-diepoxybutane |
| XL-4 | bis-or poly-epoxy |

The multiply cross linking process is akin to a discrete or digital process where the HA is first cross-linked, then the result is cross-linked a second time, then third cross-linked is done, thus forming serial cross-linking additions. This discrete or digital process is in contrast to the conventional continuous process. In one embodiment, the IPN center can be where ever relative aqueous front exists.

It should be mentioned that for the purpose of HA longevity, the more hydrophobic a cross linker is the better because hydrolysis is not favored. Sterically hindered cross linker is also preferred for the same reason mentioned. However, hydrophobicity in this case will make the HA polymer less biocompatible and will likely illicit unwanted foreign body reactions. The type of cross linker used for any part of the process will also make a difference in longevity, biocompatibility and physical properties. Application requirement will dictate the ideal polymer composition that gives the balance of properties.

Through the serial cross-linking steps, the cross-linked HA (hyaluronic acid) molecular macro structures are interpenetrated cross-linked highest at the surface that is interfacing the basic aqueous media and lowest toward the center core. After the initial cross-linking reaction step, the cross-linked HA chains lost significant mobility. Thus, an IPN (interpenetrating network) polymer is formed readily with subsequent sequential cross-linking reactions.

The rheology of cross-linked HA may be characterized as having non-Newtonian fluid behaviors. According to theory in regards mixing in a two-dimensional cavity flow, the key to effective mixing lies in producing repetitive stretching and folding, an operation referred to as a “horse-shoe-map”. The mixing can be done as a mixing of viscous Newtonian and non-Newtonian fluids, as described by Chavan et al, “Mixing of Viscous Newtonian and Non-Newtonian Fluids, pp 211-252, the content of which is incorporated by reference. Alternatively, the mixing can be done according to “Stretching and mixing of non-Newtonian fluids in time-periodic flows”, by Paulo E. Arratia, the content of which is incorporated by reference. The scaling of the mixing processes can be done as described in Wilkens et al. “How to Scale Up Mixing Processes in Non-Newtonian Fluids”, the content of which is incorporated by reference.

In the end product, the cross-linking level is non-uniform throughout the HA polymer matrix, the polymer chains become bi-axially oriented. The orientation is the result of the polar medium the HA polymer resides in.

Various implementations of mixing the reactants are described below:
1. Manual Mixing—In this example, as shown in the schematic drawing labeled FIG. 4, the mixing is manual. This method is easy to assemble and does not require expensive equipment. The type of cross-linker* and the amount** of cross-linker used at various steps may be optimized for the desired property.
   a. Dissolve HA in sodium hydroxide (reaction reactivity increases with higher pH)
   b. Cross-linking reaction
      i. Add cross-linker
      ii. Mechanically mix the mixture
      iii. The number of cross-linking steps may varied according to the desired physical property
   c. Repeat step “b” with the same cross-linker and/or another type of cross-linker. The amount of cross-linker may be the same or not the same. That depends on the physical property desired.
   d. Reaction product purification—It is important to clean up the reaction product to remove processing aides, unreacted reactants and impurities so that the main product can perform its function without interference.

Since all of the components used in the reaction are water soluble, and the reaction product is not water soluble, the cross-linked HA polymer can be purified using DI water. Furthermore, water will swell the reaction product many folds which allows the impurities to easily diffuse out of the polymer and be eliminated. Continuous flushing with DI water will speed up the purification process, and effectively rid the reaction product of unwanted impurities.

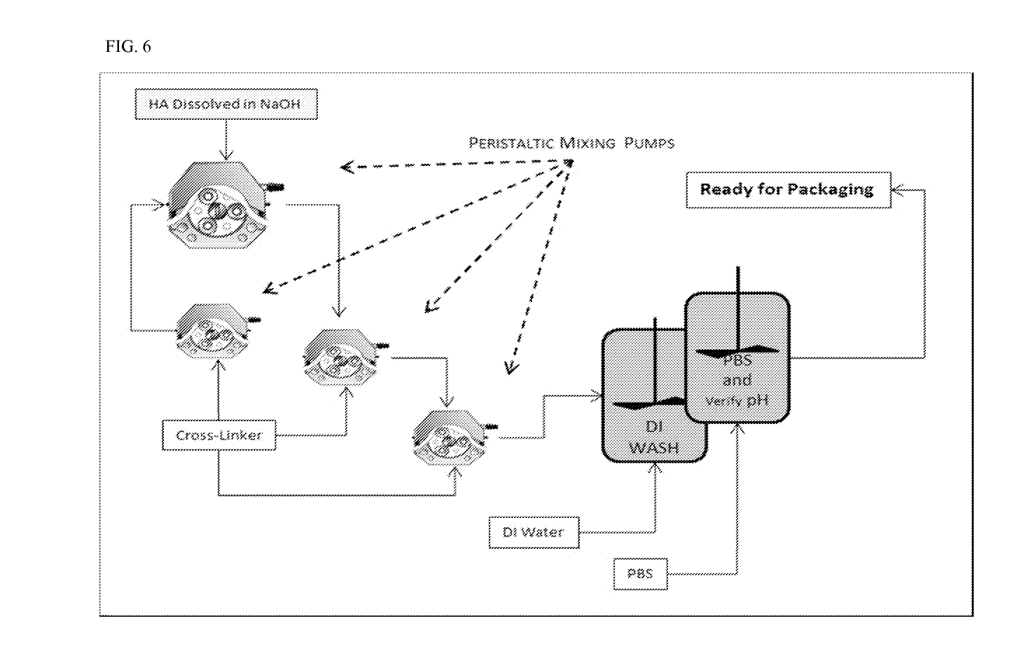

The pH of the water before mixing with the cross-linked HA and after mixing with the cross-linked HA is a good indirect indicator of the cleansing effectiveness along the process. The of the water pH before and after should not significantly changed.
e. Equilibrate in phosphate buffered saline and balance the pH to about 7.4—Drain all the water from the cross-linked HA. Add fresh PBS to at least three times the volume of the cross-linked HA and let solution mixture equilibrate for couple hours. Repeat the process another two times until the pH is about 7.4±0.7.
2. Mechanical Pressure Pumps—This approach is shown in FIG. 5, and the advantage of this method is its continuous nature. The advantage of continuous processing is the flexibility in the amount of product that can be produced. There is an upper limit, but it is unlike that of fixed quantity batch process.
   a. HA is dissolved in NaOH; the concentration of the NaOH has direct effect the reactivity OH terminals of the HA polymer chain.
   b. The cross-linking reaction takes place as soon as the HA in NaOH solution is exposed to the cross-linker
      i. The type of cross-linker may be varied
      ii. The amount of cross-linker may be varied
   c. It is important to the reproducibility of the end product that the mixing takes place quickly and efficiently.
      i. The mixing in this method takes place when the reaction mixture travels between the various changing pipes inside diameters, as shown in FIG. 2.
      ii. The number of times that the reaction mixture will go the mixing pipe apparatus can be optimized so that the desire physical property is achieved.
3. Mechanical Peristaltic Pumps—The approach is shown in FIG. 6, and mixing component is different in this method as compare to the mechanical pressure method. In the mechanical pressure method, the mixing mechanism is the mixing pipes apparatus (FIG. 1), and in the peristaltic pumps method, the mixing mechanism is in the rollers (also the pumping mechanism). Otherwise, the other features are the same for methods of FIGS. 5 and 6.
4. Cross-linked HA with Biaxial Orientation of the Molecular Macrostructure
   a. Use of Surfactants
      In this embodiment, HA molecules have several hydroxy terminals that could be reactive hydroxyl in an alkaline medium such as readily forming ether linkages with vinyl functionally terminated molecules, makes many HA modification products simple single step reactions. For example, a more hydrophobic cross-linker offers HA even better protection from degradation. These can be aliphatic diacrylates such as and the like:
         1,4-butanediol dimethacrylate,
         1,4-butanediol diacrylate,
         1,6-hexanediol diacrylate,
         1,6-hexanediol dimethacrylate,
         Ethylene glycol dimethacrylate
         Ethylene glycol diacrylate
         Poly(ethylene glycol)* diacrylate
         Poly(ethylene glycol)* dimethacrylate
      Where * indicates various molecular weight polyethylene glycol species
      These being hydrophobic cross-linkers are typically not water miscible/soluble. A surfactant is required to create a favorable environment for the molecules to come together and create a chemical reaction. Since a spectrum of polarity is being created mediated by the surfactant and the medium, the cross-linked HA product is highly biaxial oriented. The orientation of the polar and non-polar molecular macrostructure follows that of the polarity of medium that the product is resting in.
      The opposing molecular macrostructures migrate away from the medium surfaces and congregate in the center core of the molecule. In this case, the more hydrophobic interpenetrating cross-linked HA network. The molecule preserves the original softness and bio-compatibility.
      Furthermore, often the molecular macrostructure the cross-linked HA is oriented toward neighboring molecular species with similar physical properties. This feature is unique in that the physical property compliant is self adjusting.
   b. Use of Independent Pre-Cross-linked HA

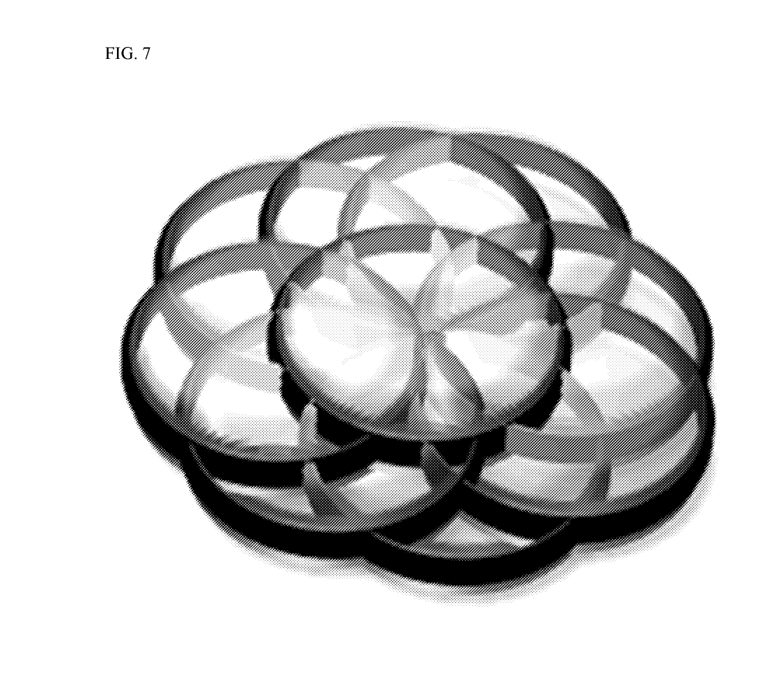

FIG. 7 shows a schematic representation of another embodiment of IPN HA and where the circles cross, IPN is formed. In one embodiment, the process is as follows:
i. Select a homogeneously cross-linked HA polymer (Poly A), and another homogeneous x-linked HA polymer (Poly B). Both are low molecular weights. There can even be a Poly C, D or E.
ii. Soak each in its theta solvents
iii. Combine the various cross-linked HA polymer mixtures. The polymer chains from A, B and so on should migrate, intertwine and become entangled at the surfaces where they interface.
iv. Add a cross-linker that has affinity for the interface and kick-off the reaction.
v. The hard core can be mechanically created using macro-molecular manipulation technique.

The other methods, used for characterization of the products according to one embodiment are described in the following examples which illustrate preferred embodiments of one embodiment without, however, being a limitation thereof. Variations and modifications can, of course, be made without departing from the spirit and scope of the invention. For example, the HA can be used as facial fillers, dermal fillers, butt fillers, breast fillers, and other body part fillers. The implants of the present invention further can be instilled, before or after implantation, with indicated medicines and other chemical or diagnostic agents. Examples of such agents include, but are not limited to, antibiotics, chemotherapies, other cancer therapies, brachy-therapeutic material for local radiation effect, x-ray opaque or metallic material for identification of the area, hemostatic material for control of bleeding, growth factor hormones, immune system factors, gene therapies, biochemical indicators or vectors, and other types of therapeutic or diagnostic materials which may enhance the treatment of the patient.

Advantages of one IPN embodiment can include one or more of the following. A natural feel is achieved through viscoelastic harmony of properties between the existing tissue and the implant. This can be done by manipulating the viscous component of the implant through flow properties by way of the particle size and particle size distribution ratios. The elastic component is intrinsic within the material tertiary structure (molecular weight and steric hindrance) and cross linking densities. The interpenetrating polymer network hydrogels have a number of desirable properties. These properties include high tensile strength with high water content, making the interpenetrating polymer network hydrogels excellent for use in dermal filling applications. Other advantages and features include: longevity without touch up, hyper-volumic degradation, anatomic compliant and iso-osmotic controlled, among others.

The present invention has been described particularly in connection with a breast, butt, or body implant, but it will be obvious to those of skill in the art that the invention can have application to other parts of the body, such as the face, and generally to other soft tissue or bone. Accordingly, the invention is applicable to replacing missing or damaged soft tissue, structural tissue or bone, or for cosmetic tissue or bone replacement.

Although the present invention has been described in relation to particular embodiments thereof, many other variations and modifications and other uses will become apparent to those skilled in the art. It is preferred, therefore, that the present invention be limited not by the specific disclosure herein, but only by the appended claims. The other methods, used for characterization of the products according to one embodiment are described in the following examples which illustrate preferred embodiments of one embodiment without, however, being a limitation thereof. Variations and modifications can, of course, be made without departing from the spirit and scope of the invention.

What is claimed is:

1. A method for forming a biocompatible cross-linked polymer having an interpenetrating polymer network (IPN), comprising:
cross-linking a heteropolysaccharide to form a single cross-linked material; and
performing one or more additional cross-linkings on the single cross-linked material to form a multiple cross-linked material,
wherein the multiple cross-linked material has one or more IPN regions resisting biodegradation in a human body than the single cross-linked material and one or more single cross-linked extensions radiating out from the IPN, wherein the combination of the IPN and the extension provide one or more of: biodegradation resistance, soft touch feeling, ease of insertion into the human body.

2. The method of claim 1, comprising injecting the biocompatible cross-linked polymer in a minimally invasive manner.

3. The method of claim 1, comprising dermally injecting the biocompatible cross-linked polymer in a minimally invasive manner.

4. The method of claim 1, comprising using a syringe to inject the biocompatible cross-linked polymer under the skin in a minimally invasive manner.

5. The method of claim 1, comprising using a syringe to inject the biocompatible cross-linked polymer in a breast or a buttock or under soft tissue in a minimally invasive manner.

6. The method of claim 1, comprising using a mechanical pump to inject the biocompatible cross-linked polymer under soft tissue in a minimally invasive manner.

7. The method of claim 1, wherein the polymer comprises one of: collagens, hyaluronic acids, celluloses, proteins, saccharides, an extracellular matrix of a biological system.

8. The method of claim 1, wherein the polymer comprises a thermoplastic, comprising converting the polymer to a thermoset.

9. The method of claim 1, comprising using cross linkers and forming thermoset polymers or to form cross linked copolymers by crosslinking with other polymer species using multifunctional monomers.

10. The method of claim 1, comprising implanting a composition with a biocompatible viscoelastic gel slurry comprising a two phase mixture, a first phase being a particulate biocompatible gel phase, said gel phase comprising a chemically cross-linked glycosaminoglycan, or said glycosaminoglycan chemically co-cross-linked with at least one other polymer selected from the group consisting of polysaccharides and proteins, said gel phase being swollen in a physiologically acceptable aqueous medium and being uniformly distributed in the second phase, said second phase comprising a polymer solution of a hydrophilic biocompatible polymer selected from the group consisting of polysaccharides, polyvinylpyrrolidone and poly ethyleneoxide in said physiologically acceptable aqueous medium, and wherein the polymer solution in the two phase mixture constitutes from 0.01 to 99.5% and the gel phase constitutes the remainder into a part of a living body where such augmentation is desired.

11. The method of claim 1, comprising adding a substance to the composition for biocompatibility

12. The method of claim 1, comprising controlling drug releases at predetermined timing according physiological events.

13. The method of claim 1, comprising carrying the drug by biocompatible and biodegradable polymers.

14. The method of claim 1, comprising dispensing the drug uniformly throughout a material matrix of the biodegradable polymer.

15. The method of claim 1, comprising housing the drug in a core-shell structure and drug release is based on diffusion and solubility.

16. The method of claim 1, comprising providing a polymer that carries the drug including one of: polylactide (PLA), polyglycolide (PGA) and copolymers of PLA/PGA tailored to meet mechanical performance and resorption rates required for applications ranging from non-structural drug delivery polymer applications to biodegradable screws or anchors.

17. The method of claim 1, comprising releasing drug into a biological environment at the same rate as a polymer rate of degradation and the rate of drug diffusing from a polymer matrix.

18. The method of claim 1, comprising blending a drug carrier polymer composition and a filler polymer composition at a predetermined ratio.

19. The method of claim 1, comprising adding one or more of: an anesthetics, a lidocaine, a compound to reduce or eliminate acute inflammatory reactions, or a composition selected from the group consisting of steroids, corticosteroids, dexamethasone, triamcinolone.

20. The method of claim 1, comprising providing a slow release substance or a fast releasing substance.

21. A composition, comprising:
a first portion of a first polymer with lightly cross-linking;
a second portion of polymer with a first serially cross-linked center overlapping the first portion and one or more lightly cross-linked extensions adjacent the serially cross-linked center; and
a third portion of polymer with a second serially cross-linked center overlapping the second portion and one or more lightly cross-linked extensions adjacent the serially cross-linked center; wherein the lightly cross-linked extensions enable the composition to be injected through a small gauge needle and the second serially cross-linked center is resistant to absorption by biological processes.

22. The composition of claim 21, wherein the polymer comprises one of: collagens, hyaluronic acids, celluloses, proteins, saccharides, an extracellular matrix of a biological system.

23. The composition of claim 21, wherein the polymer comprises one of: free radical scavengers and/or antioxidants and/or vitamins and/or enzyme inhibitor
